(19) United States
(12) Patent Application Publication
(10) Pub. No.: US 2015/0140059 A1
(43) Pub. Date: May 21, 2015

(54) NOVEL OXYSTEROL ANALOGUE, OXY149, INDUCES OSTEOGENESIS AND HEDGEHOG SIGNALING AND INHIBITS ADIPOGENESIS

(71) Applicants: The Regents of the University of California, Oakland, CA (US); University Louisville Research Foundation, Inc., Louisville, KY (US)

(72) Inventors: Farhad Parhami, Los Angeles, CA (US); Michael Jung, Los Angeles, CA (US); Frank Stappenbeck, San Diego, CA (US); William Pierce, Louisville, KY (US); K. Grant Taylor, Louisville, KY (US); Kevyn E. Merten, Louisville, KY (US)

(21) Appl. No.: 14/399,105

(22) PCT Filed: Mar. 15, 2013
(86) PCT No.: PCT/US13/32650

§ 371 Date: Nov. 5, 2014

Related U.S. Application Data
(60) Provisional application No. 61/643,776, filed on May 7, 2012.

(51) Int. Cl. C07J 41/00; A61K 45/06; C12N 5/077; A61L 31/16; A61L 31/08; A61K 31/575; A61K 35/28

(52) U.S. Cl. CPC C07J 41/0055; A61K 31/575; A61K 45/06; A61K 35/28; A61L 31/16; A61L 31/08; C12N 5/0654; A61K 48/00
USPC 424/423; 552/542; 514/182; 514/171; 424/93.7; 514/11.8; 424/676; 514/8.5; 514/8.9; 514/8.8; 514/8.6

(57) ABSTRACT

This invention relates, e.g., to a synthetic compound, Oxy149, having the structure (Formula I) or a bioactive or pharmaceutical composition comprising Oxy149 and a pharmaceutically acceptable carrier. Methods are also disclosed for using the compound or bioactive or pharmaceutical composition to treat a variety of disorders, including e.g. bone disorders, obesity, cardiovascular disorders, and neurological disorders. Oxy149 can be delivered either locally or systemically.

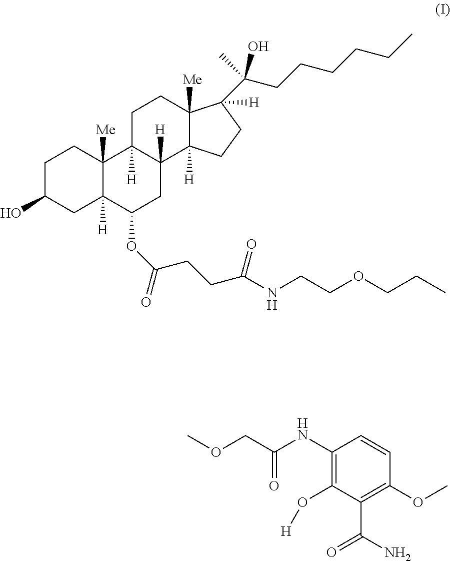

This application claims the benefit of the filing date of U.S. provisional application 61/643,776, filed May 7, 2012, which is incorporated by reference herein in its entirety.

This invention was made with Government support of Grant No. AR059794, awarded by the National Institutes of Health. The government has certain rights in the invention.

### BACKGROUND INFORMATION

Biologics are commonly employed to promote bone growth in medical applications including fracture healing and surgical management of spinal disorders (1-4). Spine fusion is often performed by orthopedic surgeons and neurosurgeons alike to address degenerative disc disease and arthritis affecting the lumbar and cervical spine. Historically, autogenous bone graft, commonly taken from the iliac crest of the patient, has been used to augment fusion between vertebral levels. However, the associated donor site morbidity, increased operating time, and increased blood loss associated with harvesting autogenous bone graft (5-7) has provided incentive to find a safe and effective alternative.

Recombinant human bone morphogenetic protein-2 (rhBMP-2) is commonly used to promote spine fusion in humans. Its use was approved in 2002 by the US Food and Drug Administration (FDA) for single-level anterior lumbar interbody fusion (8). The use of rhBMP-2 has increased significantly since this time and indications for its use have expanded to include posterior lumbar spinal fusion as well cervical spine fusion. Despite the efficacy of rhBMP-2, recent reports have called into question its safety when employed during spine fusion surgery. Reported complications have included seroma formation, soft tissue swelling, vertebral osteolysis, ectopic bone formation, retrograde ejaculation, and carcinogenicity (9-12). Moreover, airway edema has been observed with its use in the cervical spine, prompting the FDA to issue a Public Health Notification warning for its use in cervical spine operations. To date no suitable alternative has been identified that would have similar efficacy in inducing fusion without the adverse effects of rhBMP-2 (12).

Oxysterols form a large family of oxygenated derivatives of cholesterol that are present in the circulation, and in human and animal tissues. Oxysterols have been found to be present in atherosclerotic lesions and play a role in various physiologic processes, such as cellular differentiation, inflammation, apoptosis, and steroid production. Some of the present inventors previously reported that specific naturally occurring oxysterols have robust osteogenic properties (13). The most potent osteogenic naturally occurring oxysterol, 20(S)-hydroxycholesterol (“20S”) (14), is both osteogenic and anti-adipogenic when applied to multipotent mesenchymal cells capable of differentiating into osteoblasts and adipocytes. Structural modifications of 20S were previously performed to synthesize more potent analogues of 20S including Oxy34 and Oxy49, which were shown to induce the osteogenic and inhibit the adipogenic differentiation of bone marrow stromal cells (MSC) through activation of Hedgehog (Hh) signaling (15). Additionally, Oxy34 and Oxy49 stimulate spine fusion in vivo in a rat model of posterolateral spine fusion (15). Prior oxysterol molecules have properties that vary widely and unpredictably. There remains a need for new improved oxysterols as compared with rhBMP-2 and prior oxysterols, to provide increased potency and enhanced efficacy, and ease of synthesis and lower production cost. A new oxysterol could make a more feasible clinical option for physicians treating, for example, long bone fractures, spine disorders, and osteoporosis.

The osteogenic oxysterols described above are particularly useful for direct, localized administration to target cells, tissues or organs of interest. Currently, there are no commercial anabolic agents for systemic delivery and intervention in bone disorders, e.g., osteoporosis, which is a disease in which bone is lost during aging in both men and women and after menopause in women. The only currently available systemically delivered agent that induces bone formation is Forteo® (teriparatide [rDNA origin] injection), which is expensive, has adverse effects and is FDA approved for use no longer than 24 months. There is a need for an osteogenic agent, such as an osteogenic oxysterol, which is safer and more effective for induction of systemic bone formation following systemic administration in, e.g., osteoporotic patients.

### DESCRIPTION OF THE DRAWINGS

FIG. 1 shows the molecular structures of osteogenic oxysterols. The molecular structures of 20(S)-hydroxycholesterol (20S), Oxy34, Oxy49, and Oxy133 are shown. Oxy34 is different from 20S in having an extra OH group on C6 and the double bond between C5 and C6 is eliminated. Oxy49 has a similar structure to Oxy34 and includes a double bond between C25 and C27. Oxy133 differs from Oxy34 and 49 by the deletion of C27 and increasing the length of the side chain by one carbon.

FIG. 2 shows the dose-dependent activation of alkaline phosphatase activity by oxysterols. (FIG. 2A) C3HT101/2 cells or (FIG. 2B) M2-10B4 cells at confluence were treated with control vehicle or 0.125-10 μM of Oxy133. For direct comparison to Oxy133, C3H cells were also treated with Oxy34 and Oxy49 (FIG. 2A). After 4 days, alkaline phosphatase (ALP) activity was measured in whole cell extracts. Data from a representative of three separate experiments are reported as the mean of triplicate determinations+SD and normalized to protein concentration. (p<0.0001 for cells treated with 0.25 μM or higher dose of all oxysterols vs. control vehicle treated cells).

FIG. 3 shows that oxy133 induces osteogenic differentiation. (FIG. 3A) C3HT101/2 cells at confluence were treated with control vehicle or 2.5 μM Oxy133 in osteogenic media. Expression of osteogenic genes Runx2, ALP, BSP, OSX, and OCN was measured by quantitative real-time PCR after 48 hours (48 h), 4, 7, and 14 days of treatment. Results from a representative experiment are reported as the mean of triplicate determinations±SD. (p<0.005 for control vs. Oxy133 at all time points for ALP, BSP and OSX and at 4, 7, and 14 days for Runx2 and OCN). (FIG. 3B) C3H10T1/2 cells were treated with control vehicle or 2.5 μM Oxy133 for 3 weeks. To examine extracellular mineralization von Kossa staining was performed and mineralized matrix appears as dark black staining under light microscopy (10×). (FIG. 3C) In parallel cultures to those described in (B), mineralization was quantified using a 45Ca incorporation assay (p<0.005 for control vs. all concentrations of Oxy133). (FIG. 3D) Primary human MSC were treated in osteogenic medium with control vehicle or 5 μM Oxy133 for 4 weeks. Expression of osteogenic genes OSX, BSP, and OCN was measured by quantitative real-time PCR. Results from a representative experiment are reported as the mean of triplicate determination±SD (p<0.05 for all genes in control vs. Oxy133 treated cells). (FIG. 3E) Primary human MSC were treated in osteogenic medium with control vehicle or 0.5, 1, and 5 μM Oxy133 for 5 weeks. To examine extracellular mineralization von Kossa staining was performed and mineralized matrix appears as dark black staining under light microscopy (10×).

FIG. 4 shows the role of Hedgehog pathway in Oxy133-induced osteogenic differentiation. (FIG. 4A) C3H10T1/2 cells at confluence were treated in osteogenic medium with control vehicle or Oxy133 in the presence or absence of 4 μM cyclopamine (Cyc). After 4 days ALP activity, and after 7 days the expression of osteogenic genes ALP, BSP, and OSX was measured by quantitative real-time PCR (p<0.001 for control vs. Oxy133, and for Oxy133 vs. Oxy133+Cyc for ALP activity and for the expression of all genes shown). (FIG. 4B) C3H10T1/2 cells were transfected with control plasmid (pGL3b) or a plasmid containing 8X-Gli luciferase reporter and treated with control vehicle or Oxy133, and luciferase activity was determined after 48 hours. Results from a representative experiment are reported as the mean of triplicate determinations±SD. (p<0.001 for control vs. Oxy133 at 100 nM, 250 nM, and 1 μM Oxy133). (FIG. 4C) The amount of YFP-Smo captured by 20S beads or control beads was compared in samples containing either no competitor or 50 μM of a free competitor sterol (20S, Oxy133 or Oxy16). The YFP-Smo captured by the beads was measured by Western blot (top) and plotted (bottom) relative to the amount captured in the binding reaction with no competitor.

FIG. 5 shows plain radiographs of fusion masses formed by BMP2 and Oxy133. Faxitron images of two representative animals from the indicated groups at 8 weeks postoperatively are shown. Arrowheads signify lack of bone formation; arrows signify bone formation. Group I (Control); intertransverse process space with no bone formation. Group II (BMP2); bridging bone mass and bilateral fusion at L4-L5. Group III (Oxy133-20 mg); bridging bone mass and bilateral fusion at L4-L5. Group IV (Oxy133-2 mg); bridging bone mass and bilateral fusion at L4-L5 in animals that showed induction of fusion by Oxy133.

FIG. 6 shows microCT of fusion masses formed by BMP2 and Oxy133. Micro CTs of two representative animals from the indicated groups are shown. Arrowheads signify lack of bone formation; arrows signify bone formation. Group I (Control); intertranverse process space with no bone formation. Group II (BMP2); bone mass bridging the intertransverse process space and bilateral fusion at L4-L5. Group III (Oxy133-20 mg); bone mass bridging the intertransverse process space and bilateral fusion at L4-L5. Group IV (Oxy133-2 mg); bone mass bridging the intertransverse process space and bilateral fusion at L4-L5 in animals that showed induction of fusion by Oxy133. Group V (Oxy133-0.2 mg); arrow on the far right indicates a small amount of bone formation from the L5 transverse process.

FIG. 7 shows histological analysis of the effect of Oxy133 on spinal fusion. (FIG. 7A) Coronal histological sections of two separate representative animals from each group are shown (10×). Group I (Control) has no significant bone formation at the intertransverse process space (arrowheads). Group II (BMP2) demonstrates bridging bone at L4-L5 (arrows) with clear evidence of trabecular and cortical bone forming the fusion mass. Group III (Oxy133-20 mg) and Group IV (Oxy133-2 mg) specimens demonstrate significant bone formation at the intertransverse process space (arrows) with trabecular and cortical bone formation comparable to that induced by BMP2. (FIG. 7B) Coronal histological sections from two animals each in Groups II (BMP2) and Group III (Oxy133-20 mg) demonstrate significant adipocyte formation in the fusion mass of BMP2 treated animals and substantially fewer adipocytes in the fusion mass from oxysterol treated animals (arrows, magnification 20×).

FIG. 8 shows that the osteogenic differentiation marker, alkaline phosphatase activity, is induced by Oxy133 and Oxy149 in (FIG. 8A) M2-10B4 bone marrow stromal cells and in (FIG. 8B) C3H10T1/2 embryonic fibroblast. Cells at confluence were treated with vehicle, Oxy133 or Oxy149. After 4 days, alkaline phosphatase (ALP) activity was measured in whole cell extracts. Data from a representative of three separate experiments are reported as the mean of triplicate determinations SD and normalized to protein concentration.

FIG. 9 shows that Oxy133 and Oxy149 induce osteogenic differentiation and the expression of osteogenic differentiation marker genes. C3HT101/2 cells at confluence were treated with vehicle, Oxy133 or Oxy149 in osteogenic media. Expression of osteogenic genes Runx2 (FIG. 9E), ALP (FIG. 9A), bone sialoprotein (BSP) (FIG. 9B), Osterix (OSX) (FIG. 9C), and Osteocalcin (OCN) (FIG. 9D) was measured by quantitative real-time PCR after 8 days of treatment. Results from a representative experiment are reported as the mean of triplicate determinations±SD.

FIG. 10 shows that Oxy133 and Oxy149 induce Hedgehog pathway signaling. C3H10T1/2 cells at confluence were treated in osteogenic medium with control vehicle, Oxy133, or Oxy149 in the presence or absence of 4 μM cyclopamine (Cyc). After 72 hours the expression of Hedgehog pathway target genes Gli1 (FIG. 10A), Ptch1 (FIG. 10B), and HIP (FIG. 10C) was measured by quantitative real-time PCR. Results from a representative experiment are reported as the mean of triplicate determinations±SD.

### DESCRIPTION

The present inventors describe and characterize herein a molecule (compound) which is a hybrid between a newly identified, particularly effective, oxysterol molecule (Oxy133) and a tetracycline-derived bone targeting moiety. The hybrid molecule is called Oxy149. Because of its ability to be delivered selectively and specifically to bone, Oxy149 is particularly suitable for systemic delivery to a subject, e.g. for targeting osteoporosis.

The present inventors first identify herein an osteogenic oxysterol, Oxy133 which is well-suited for a variety of clinical uses, and describe its ability to promote osteogenic differentiation in vitro and spine fusion in a rat model in vivo. Of the large number of oxysterol analogues synthesized and tested, Oxy133 was unexpectedly particularly effective and easy to synthesize. Oxy133 induced significant expression of osteogenic markers Runx2, osterix (OSX), alkaline phosphatase (ALP), bone sialoprotein (BSP), and osteocalcin (OCN) in C3H10T1/2 mouse embryonic fibroblasts. Oxy133-induced activation of an 8X-Gli luciferase reporter, its direct binding to Smoothened, and the inhibition of Oxy133-induced osteogenic effects by the Hedgehog (Hh) pathway inhibitor, cyclopamine, demonstrated a role of the Hh pathway in mediating osteogenic responses to Oxy133. In addition, Oxy133 induced the expression of OSX, BSP, and OCN and stimulated robust mineralization in primary human mesenchymal stem cells. In vivo, bilateral spine fusion in animals treated with Oxy133 at the fusion site was observed on X-ray after only 4 weeks and confirmed with manual assessment, micro-CT, and histology after 8 weeks, with equal efficiency to bone morphogenetic protein-2 (BMP2). However, unlike BMP2, Oxy133 did not induce adipogenesis in the fusion mass and resulted in the formation of denser bone as evidenced by greater BV/TV ratio and smaller trabecular separation. Oxy133 is thus useful for treating conditions that would benefit from localized stimulation of bone formation, including. e.g, spine fusion, fracture repair, bone regenerative/tissue engineering applications, augmentation of bone density in the jaw for dental implants, osteoporosis or the like.

The inventors also demonstrate that Oxy133 inhibits adipogenesis of pluripotent MSC cells. Oxy133 is thus useful for treating conditions such as, e.g., xanthoma formation, localized accumulation of fat pads and obesity.

Advantages of Oxy133 include, e.g., greater ease of synthesis and improved time to fusion when compared to other osteogenic oxysterols studied by the inventors.

Furthermore, the inventors describe herein a modified form of Oxy133, to which is attached a tetracycline-derived molecule that functions as a bone targeting moiety. This hybrid molecule, called Oxy149, is selectively and specifically delivered to bone (selectively homes to bone) due to its linkage to the bone targeting agent. Without wishing to be bound by any particular mechanism, it is suggested that Oxy149 selectively accumulates in bone and stimulates mesenchymal stem cells to undergo osteogenic differentiation and make new bone, and that this stimulation of osteogenic differentiation is mediated through the activation of Hedgehog signaling in bone cells. Regardless of the mechanism by which it functions, Oxy149, because it is delivered selectively and specifically to bone, is effective for osteogenesis following systemic delivery to a subject. The ability to be delivered systemically represents a significant advantage, e.g. for the treatment of osteoporotic subjects. Oxy149 is a small molecule osteogenic oxysterol that can serve as a member of the next generation of bone anabolic therapeutic agents, as well as a useful agent for treatment of a variety of other conditions, including conditions which would benefit from a stimulation of a Hh pathway activity.

One aspect of the invention is a compound, named Oxy149, having the formula

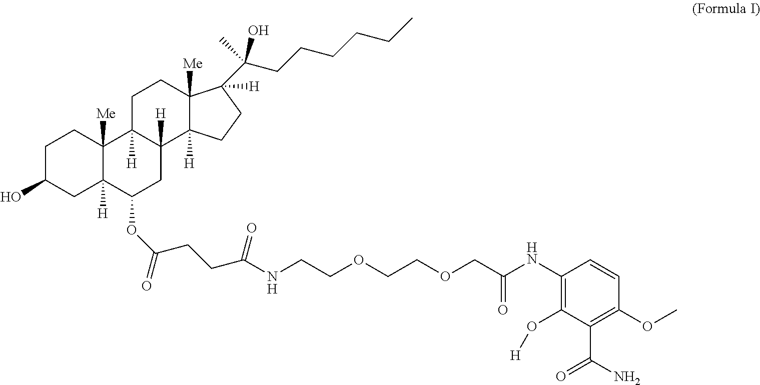

or a pharmaceutically acceptable salt or solvate thereof.

A component of Oxy149 is the oxysterol Oxy133, which has the formula

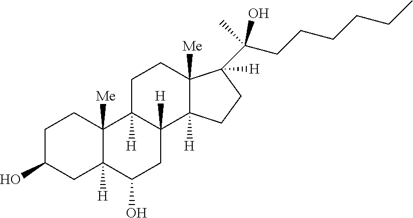

Another aspect of the invention is a bioactive or pharmaceutical composition comprising Oxy149 or a pharmaceutically acceptable salt or solvate thereof and a pharmaceutically acceptable carrier. The terms “bioactive” composition or “pharmaceutical” composition are used interchangeably herein. Both terms refer to compositions that can be administered to a subject, used to coat or be present in a medical device that is introduced into a subject, or the like. These bioactive or pharmaceutical compositions are sometimes referred to herein as “pharmaceutical compositions or bioactive compositions of the invention.” Sometimes the phrase “administration of Oxy149” is used herein in the context of administration of this compound to a subject (e.g., contacting the subject with the compound). It is to be understood that the compound for such a use can generally be in the form of a pharmaceutical composition or bioactive composition comprising the Oxy149.

Another aspect of the invention is a method for inducing (stimulating, enhancing) a hedgehog (Hh) pathway mediated response, in a cell or tissue, e.g. in a subject, comprising contacting the cell or tissue with an effective amount (e.g. a therapeutically effective amount) of Oxy149, wherein the hedgehog (Hh) pathway mediated response is the stimulation of osteoblastic differentiation, osteomorphogenesis, and/or osteoproliferation. The Hh mediated response can be useful in regenerative medicine.

Another aspect of the invention is a method for treating a subject having a bone disorder, osteopenia, osteoporosis, or a bone fracture, comprising administering to the subject an effective amount of a bioactive composition or pharmaceutical composition comprising Oxy149. The subject can be administered the bioactive composition or pharmaceutical composition at a therapeutically effective dose in an effective dosage form at a selected interval to, e.g., increase bone mass, ameliorate symptoms of osteoporosis, or reduce, eliminate, prevent or treat other conditions which would benefit from an increase in osteomorphogenesis and/or osteoproliferation. The subject can be administered the bioactive composition or pharmaceutical composition at a therapeutically effective dose in an effective dosage form at a selected interval to ameliorate the symptoms of osteoporosis. In one embodiment, the subject is treated to induce bone formation by harvesting mammalian mesenchymal stem cells (e.g., from the subject or from a suitable mammal, or from a tissue or cell bank), treating the mammalian mesenchymal cells with Oxy149 to induce osteoblastic differentiation of the cells, and administering the differentiated cells to the subject.

In any of the methods of the invention, the Oxy149 can be administered to a cell, tissue or organ by local administration. For example, the Oxy149 can be applied locally with a cream or the like, or it can be injected or otherwise introduced directly into a cell, tissue or organ, or it can be introduced with a suitable medical device (e.g. an implant). Alternatively, the Oxy149 can be administered systemically, e.g., orally, intravenously (though IV) or via injection such as intraperitoneal (IP) injection.

Another aspect of the invention is a kit for carrying out one or more of the methods described herein. The kit can comprise an effective amount (e.g. a therapeutically effective amount) of Oxy149, optionally in a container.

Another aspect of the invention is an implant for use in the body of a subject (e.g., an animal such as a human) comprising a substrate having a surface. The surface or insides of the implant comprises a bioactive composition or pharmaceutical composition comprising Oxy149 in an amount sufficient to induce bone formation in the surrounding bone tissue.

Optionally, a bioactive composition, method, kit or medical device of the invention can comprise one or more other suitable therapeutic agents, such as, e.g., parathyroid hormone, sodium fluoride, insulin-like growth factor I (ILGF-I), insulin-like growth factor II (ILGF-II), transforming growth factor beta (TGF-β), a cytochrome P450 inhibitor, an osteogenic prostanoid, BMP 2, BMP 4, BMP 7, BMP 14 and/or an anti-resorptive agent such as, e.g., bisphosphonate.

### Oxy149 has the Structure

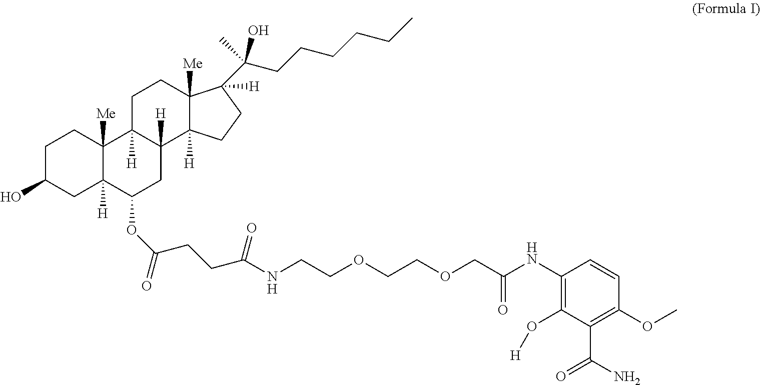

Its chemical name is (3S,5S,6S,8R,9S,10R,13S,14S,17S)-3-hydroxy-17-((S)-2-hydroxyoctan-2-yl)-10,13-dimethylhexadecahydro-1H-cyclopenta[a]phenanthren-6-yl 4-((2-(2-(2-((3-carbamoyl-2-hydroxy-4-methoxyphenyl)amino)-2-oxoethoxy)ethoxy)ethyl)amino)-4-oxobutanoate

Example II describes the design of Oxy133 and a procedure for synthesizing the molecule, as well as a synthetic procedure for linking Oxy133 to a bone targeting moiety to generate the hybrid molecule, Oxy149. The tetracycline derivative which is fused to Oxy133 to form Oxy149 was originally designed and characterized to act as a bone delivery system when linked to estradiol. See, e.g., U.S. Pat. No. 8,071,575, which is incorporated by reference herein in its entirety. The present application is directed primarily to the particular bone targeting moiety which is attached to Oxy133 to generate Oxy149. However, variants of the bone targeting portion, or variants in the linking region between the bone targeting portion and the Oxy133, as described, e.g., in U.S. Pat. No. 8,071,575, are also included.

In addition to the compound Oxy149 as shown in Formula I, other embodiments of the invention encompass any and all individual stereoisomers at any of the stereocenters shown in the Formula, including diastereomers, racemates, enantiomers, and other isomers of the compound. In embodiments of the invention, “Oxy149” or “compound having Formula I” or “Oxy149 or a pharmaceutically acceptable salt thereof” may include all polymorphs and solvates of the compound, such as hydrates and those formed with organic solvents. A “solvate” is a complex or aggregate formed by one or more molecules of a solute, e.g. a compound or a pharmaceutically-acceptable salt thereof, and one or more molecules of a solvent. Such solvates can be crystalline solids having a substantially fixed molar ratio of solute and solvent. Suitable solvents will be known by those of ordinary skill in the art, e.g., water, ethanol or dimethylsulfoxide. Such isomers, polymorphs, and solvates may be prepared by methods known in the art, such as by regiospecific and/or enantioselective synthesis and resolution.

The ability to prepare salts depends on the acidity or basicity of a compound. Suitable salts of the compound include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compounds.

It is to be understood that references herein to “Oxy149” include pharmaceutically acceptable salts or solvates thereof.

In any of the methods, compositions or kits of the invention, particularly for use in treating a subject, a composition of the invention may optionally be in combination with one or more other suitable therapeutic agents. Any therapeutic agent that is suitable for treatment of a particular condition can be used. Suitable such agents or drugs will be evident to one skilled in the art. For example, for the treatment of bone disorders, a conventional therapeutic drug can be used in combination with a composition of the invention. Some such agents include, e.g., parathyroid hormone, sodium fluoride, insulin-like growth factor I (ILGF-I), insulin-like growth factor II (ILGF-II), transforming growth factor beta (TGF-β), a cytochrome P450 inhibitor, an osteogenic prostanoid, BMP 2, BMP 4, BMP 7, BMP 14, and/or bisphosphonates or other inhibitors of bone resorption.

A composition or compound of the invention can be formulated as a pharmaceutical composition, which comprises a composition of the invention and pharmaceutically acceptable carrier. By a “pharmaceutically acceptable carrier” is meant a material that is not biologically or otherwise undesirable, i.e., the material may be administered to a subject without causing any undesirable biological effects or interacting in a deleterious manner with any of the other components of the pharmaceutical composition in which it is contained. The carrier is naturally selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18^{th} ed., Mack Publishing Company, 1990. Some suitable pharmaceutical carriers will be evident to a skilled worker and include, e.g., water (including sterile and/or deionized water), suitable buffers (such as PBS), physiological saline, cell culture medium (such as DMEM), artificial cerebral spinal fluid, dimethylsulfoxide (DMSO), or the like.

One of skill in the art will appreciate that a particular formulation of the invention will depend, at least in part, upon the particular agent or combination of agents that is employed and the chosen route of administration. Accordingly, there is a wide variation of suitable formulations of compositions of the present invention. Some representative formulations are discussed below. Others will be evident to a skilled worker. Oxy149 can be administered locally or directly to a cell, tissue or organ in need of treatment, or it can be administered systemically.

Formulations or compositions suitable for oral administration can consist of liquid solutions, such as an effective amount of Oxy149 dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract.

Formulations suitable for parenteral administration (e.g., intravenous) include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (i.e., lyophilized) condition requiring only the addition of the sterile liquid carrier, for example, water, for injections, immediately prior to use. Extemporaneous injection solutions and suspensions can be prepared from sterile powders, granules, and tablets of the kind previously described.

Oxy149, alone or in combination with other therapeutic agents, can be made into aerosol formulations to be administered via inhalation. These aerosol formulations can be placed into pressurized acceptable propellants, such as dichlorodifluoromethane, propane, nitrogen, and the like.

Suitable formulations for topical administration include lozenges comprising the active ingredient in a flavor, usually sucrose and acacia or tragacanth; pastilles comprising the active ingredient in an inert base, such as gelatin and glycerin, or sucrose and acacia; mouthwashes comprising the active ingredient in a suitable liquid carrier; or creams, emulsions, suspensions, solutions, gels, creams, pastes, foams, lubricants, sprays, suppositories, or the like.

Other suitable formulations include, e.g., hydrogels and polymers suitable for timed release of Oxy149, or nanoparticles for small dose delivery of Oxy149, Such formulations are well-known to those of skill in the art.

A person skilled in the art will appreciate that a suitable or appropriate formulation can be selected, adapted or developed based upon the particular application at hand. In addition, the pharmaceutical compositions of the present invention may be prepared for administration by a variety of different routes, whether systemic, local or both. Such examples include, but are not limited to, administrations performed intraarticularly, intracranially, intradermally, intrahepatically, intramuscularly, intraocularly, intraperitoneally, intrathecally, intravenously, subcutaneously, transdermally, or directly into a bone region atherosclerotic site, such as by direct injection, introduction with a catheter or other medical devise, topical application, direct application, and/or by implanting a device into in an artery or other appropriate tissue site.

Oxy149 may be formulated to be contained within, or adapted to release by a surgical or medical device or implant. In certain aspects, an implant may be coated or otherwise treated with Oxy149. For example, hydrogels, or other polymers, such as biocompatible and/or biodegradable polymers, may be used to coat an implant with the compositions of the present invention (i.e., the composition may be adapted for use with a medical device by using a hydrogel or other polymer). Polymers and copolymers for coating medical devices with an agent are well-known in the art. Examples of medical devises and implants include, but are not limited to, sutures and prostheses such as prosthetic joints, and can be in the shape, e.g., of a pin, screw, plate or prosthetic joint.

An “effective amount” of Oxy149, as used herein, refers to an amount that can bring about at least a detectable effect. A “therapeutically effective amount,” as used herein, refers to an amount that can bring about at least a detectable therapeutic response in a subject being treated (e.g., the amelioration of one or more symptoms) over a reasonable period of time.

In embodiments of the invention, Oxy149 can stimulate or inhibit a therapeutic response, as measured by any of a variety of conventional assays, by about 1%, 5%, 10%, 20%, 30%, 40%, 50% 150%, 200% or more of that in an untreated control sample. Intermediate values in these ranges are also included.

Dosages for Oxy149 can be in unit dosage form, such as a tablet or capsule. The term “unit dosage form,” as used herein, refers to physically discrete units suitable as unitary dosages for animal (e.g., human) subjects, each unit containing a predetermined quantity of an agent of the invention, alone or in combination with other therapeutic agents, calculated in an amount sufficient to produce the desired effect in association with a pharmaceutically acceptable diluent, carrier, or vehicle.

One skilled in the art can routinely determine the appropriate dose, schedule, and method of administration for the exact formulation of the composition being used, in order to achieve the desired effective amount or effective concentration of the agent in the individual patient. One skilled in the art also can readily determine and use an appropriate indicator of the “effective concentration” of the compounds, for example, Oxy149, by a direct or indirect analysis of appropriate patient samples (e.g., blood and/or tissues), in addition to analyzing the appropriate clinical symptoms of the disease, disorder, or condition.

The exact dose of Oxy149 or composition thereof administered to an animal, such as a human, in the context of the present invention will vary from subject to subject, depending on the species, age, weight and general condition of the subject, the severity or mechanism of any disorder being treated, the particular agent or vehicle used, its mode of administration, other medications the patient is taking and other factors normally considered by an attending physician, when determining an individual regimen and dose level appropriate for a particular patient, and the like. The dose used to achieve a desired concentration in vivo will be determined by the potency of the form of the Oxy149, the pharmacodynamics associated with the Oxy149 in the host, with or without additional agents, the severity of the disease state of infected individuals, as well as, in the case of systemic administration, the body weight and age of the individual. The size of the dose may also be determined by the existence of any adverse side effects that may accompany the particular agent, or composition thereof, employed. It is generally desirable, whenever possible, to keep adverse side effects to a minimum.

For example, a dose can be administered in the range of from about 5 ng (nanograms) to about 1000 mg (milligrams), or from about 100 ng to about 600 mg, or from about 1 mg to about 500 mg, or from about 20 mg to about 400 mg. For example, the dose can be selected to achieve a dose to body weight ratio of from about 0.0001 mg/kg to about 1500 mg/kg, or from about 1 mg/kg to about 1000 mg/kg, or from about 5 mg/kg to about 150 mg/kg, or from about 20 mg/kg to about 100 mg/kg. For example, a dosage unit can be in the range of from about 1 ng to about 5000 mg, or from about 5 ng to about 1000 mg, or from about 100 ng to about 600 mg, or from about 1 mg to about 500 mg, or from about 20 mg to about 400 mg, or from about 40 mg to about 200 mg of Oxy149 or a composition comprising Oxy149. In one embodiment of the invention, amounts of Oxy149 as above (e.g., a few grams) are administered locally, such as. in a spine fusion procedure as part of a scaffold.

A dose can be administered once per day, twice per day, four times per day, or more than four times per day as required to elicit a desired therapeutic effect. For example, a dose administration regimen can be selected to achieve a blood serum concentration of a compound of the present invention in the range of from about 0.01 to about 1000 nM, or from about 0.1 to about 750 nM, or from about 1 to about 500 nM, or from about 20 to about 500 nM, or from about 100 to about 500 nM, or from about 200 to about 400 nM. For example, a dose administration regime can be selected to achieve an average blood serum concentration with a half maximum dose of a compound of the present invention in the range of from about 1 μg/L (microgram per liter) to about 2000 μg/L, or from about 2 μg/L to about 1000 μg/L, or from about 5 μg/L to about 500 μg/L, or from about 10 μg/L to about 400 μg/L, or from about 20 μg/L to about 200 μg/L, or from about 40 μg/L to about 100 μg/L.

Certain embodiments of the invention may also include treatment with an additional agent which acts independently or synergistically with the Oxy149 to improve the therapeutic results. When given in combined therapy, the agent other than Oxy149 can be given at the same time as the Oxy149, or the dosing can be staggered as desired. The two (or more) drugs also can be combined in a composition. Doses of each can be less when used in combination than when either is used alone. Suitable doses can be determined by a skilled worker, using standard dosage parameters.

As used herein, the singular forms “a,” “an” and “the” include plural referents unless the context clearly dictates otherwise.

A “subject,” as used herein, includes any animal that exhibits a symptom of a condition that can be treated with Oxy149. Suitable subjects (patients) include laboratory animals (such as mouse, rat, rabbit, or guinea pig), farm animals, and domestic animals or pets (such as a cat, dog, or horse). Non-human primates including human patients, are included. Typical subjects include animals that exhibit aberrant amounts (lower amounts than a “normal” or “healthy” subject) of one or more physiological activities that are stimulated by Hedgehog signaling. The aberrant activities may be regulated by any of a variety of mechanisms, including activation of a Hedgehog activity. The aberrant activities can result in a pathological condition.

One embodiment of the invention is a kit useful for any of the methods disclosed herein, either in vitro or in vivo. Such a kit comprises Oxy149 or a bioactive or pharmaceutical composition thereof, and can comprise one or more other oxysterols, e.g. which result in an increase in a Hh pathway-mediated activity, or other suitable therapeutic agents. Optionally, the kits comprise instructions for performing the method. Optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form. A skilled worker will recognize components of kits suitable for carrying out any of the methods of the invention.

A variety of conditions can be treated with Oxy149, used alone or in combination with other therapeutic agents.

As shown, e.g., in the Examples herein, Oxy149 results in an increase in hedgehog pathway activity.

One effect of Oxy149 is to target pluripotent cells to induce their lineage specific differentiation into various cell types, e.g., osteoblasts, For example, as shown in the Examples, mesenchymal stem cells treated with Oxy149 showed induced expression of markers of osteoblast differentiation. Without wishing to be bound by any particular mechanism, it is suggested that this lineage specific differentiation is due to the induction of Hedgehog signaling in these cells. However, methods of treatment discussed herein are included in the present invention, regardless of the mechanism by which the Oxy149 functions. Oxy149 is useful for treating conditions which would benefit from stimulation of bone formation, osteoblastic differentiation, osteomorphogenesis and/or osteoproliferation. Among these conditions or treatments are, e.g., osteoinductive therapy for stimulation of localized bone formation in spine fusion or osteoporosis, bone fracture repair or healing, dental procedures for which increased bone formation in the jaw is of clinical benefit, repair of craniofacial bone defects induced by trauma or congenital defects such as cleft palate/lip, and a number of other musculoskeletal disorders in which native bone growth is inadequate, which will be evident to skilled workers. Treatment can be administered to treat open fractures and fractures at high risk of non-union, and in subjects with spinal disorders, including subjects in need of spine fusion (e.g., anterior lumbar interbody fusion, posterior lumbar spinal fusion, and cervical spine fusion) or subjects having degenerative disc disease or arthritis affecting the lumbar and cervical spine. Furthermore, Oxy149 can be used to treat osteoporosis, particularly in the aging and post-menopausal population, resulting from increased bone resorption by osteoclasts in parallel with decreased bone formation by osteoblasts.

More particularly, the following types of bone-related treatments can be carried out:

1. Oxy149 is used as an osteogenic agent delivered locally in the body in order to stimulate localized bone formation, using a scaffold that is composed of a compatible molecule such as but not limited to collagen I, which absorbs Oxy149 and then is placed inside the body. For example the scaffold containing Oxy149 and which can be placed in between transverse processes or in the intervertebral disc where the fusion of two or more vertebrae is indicated, for example in spine fusion, pseudoarthrosis, and non-union fusions. In other embodiments, the scaffold containing Oxy149 is placed in a fractured bone in order to simulate bone formation and healing of the fracture; is placed in a bone defect such as calvarial or maxillofacial bone defects where bone regeneration by Oxy149 is indicated; or is placed in the jaw bone in order to stimulate bone formation as a means of regenerating bone prior to dental procedures such as dental implants.

2. Oxy149 is used as an osteogenic agent in vitro For example, it is administered to osteoprogenitor cells, for example mesenchymal stem cells, in order to stimulate their osteogenic differentiation prior to the application of such cells in orthopedic and other procedures as indicated in 1) above order to stimulate localized bone formation.

3. Oxy149 is used in vitro in order to stimulate the Hedgehog signaling pathway in osteoprogenitor cells, thereby leading to the osteogenic differentiation of the cells in vitro or in vivo.

Another embodiment of the invention relates to hybrid molecules comprising Oxy133 or other osteogenic oxysterols described previously by the some of the present inventors, wherein the oxysterols are linked to other versions of tetracycline-derived bone targeting moieties described by some of the present inventors. Some such moieties are described, e.g., in U.S. Pat. No. 7,196,220 and U.S. Pat. No. 7,196,220.

Any osteogenic oxysterol molecule can be linked (conjugated) to such a tetracycline derivative and used as described herein. Representative such oxysterols include Oxy8, 34, 40 and 49, or other suitable oxysterols previously described by the present inventors or by others. Some such hybrid molecules include the following:

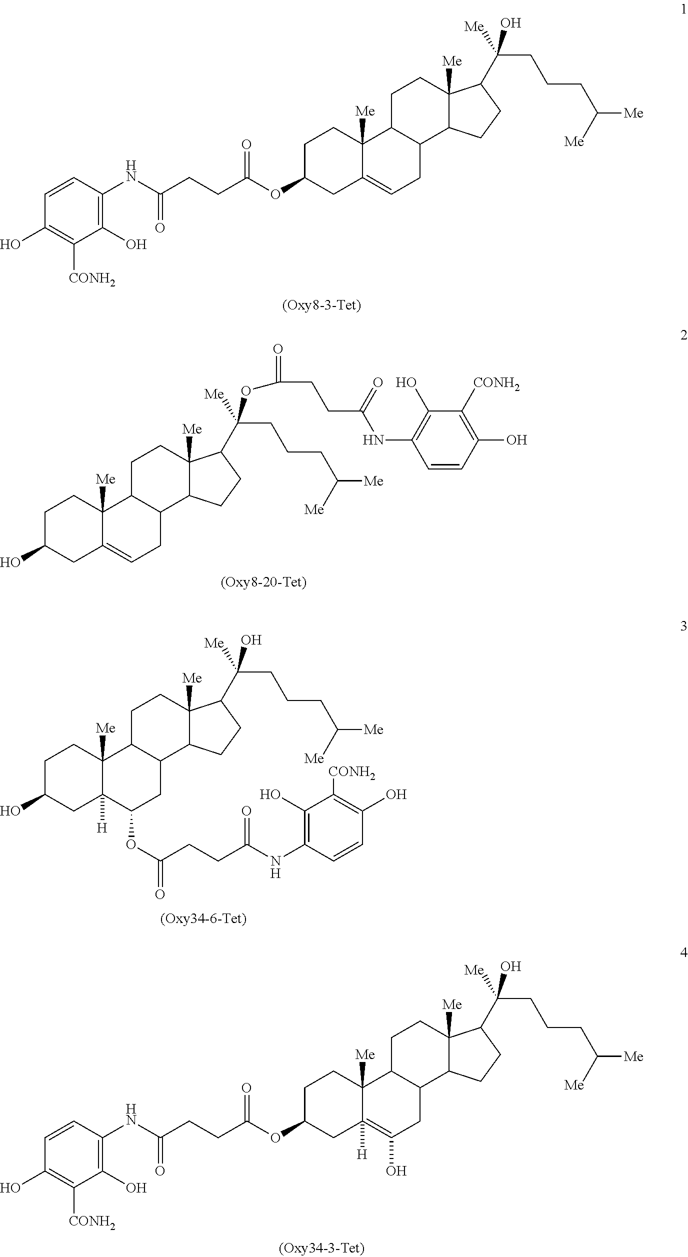

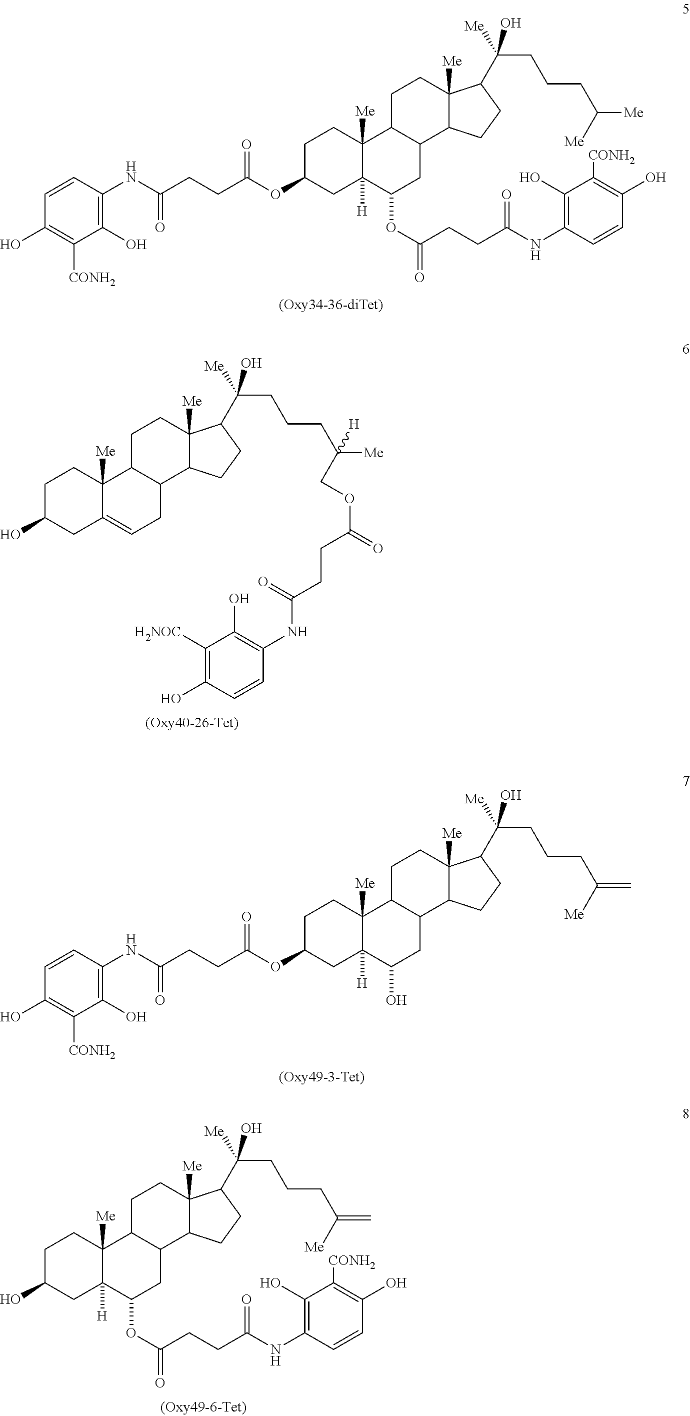 [SMILES: CC(C)CCC[C@](C)(O)C1CCC2C3C[C@H](OC(=O)CCC(=O)Nc4ccc(O)c(C(N)=O)c4O)[C@H]4C[C@@H](OC(=O)CCC(=O)Nc5ccc(O)c(C(N)=O)c5O)CC[C@]4(C)C3CC[C@@]21C ; CC(CCC[C@](C)(O)C1CCC2C3CC=C4C[C@@H](O)CC[C@]4(C)C3CC[C@@]21C)COC(=O)CCC(=O)Nc1ccc(O)c(C(N)=O)c1O ; C=C(C)CCC[C@](C)(O)C1CCC2C3C[C@H](O)[C@H]4C[C@@H](OC(=O)CCC(=O)Nc5ccc(O)c(C(N)=O)c5O)CC[C@]4(C)C3CC[C@@]21C ; C=C(C)CCC[C@](C)(O)C1CCC2C3C[C@H](OC(=O)CCC(=O)Nc4ccc(O)c(C(N)=O)c4O)[C@H]4C[C@@H](O)CC[C@]4(C)C3CC[C@@]21C]

In the foregoing and in the following examples, all temperatures are set forth in uncorrected degrees Celsius; and, unless otherwise indicated, all parts and percentages are by weight.

### EXAMPLES

### Example I

### Materials and Methods

### Cell Culture and Reagents

Mouse multipotent bone marrow stromal cell (MSC) line, M2-10B4 (M2), and embryonic fibroblast cell line C3H10T1/2 (C3H) were purchased from American Type Culture Collection (Rockville, Md.) and cultured as we have previously reported (14,15). Treatment to induce osteogenic differentiation was performed in RPMI for M2 cells or DMEM for C3H cells containing 5% fetal bovine serum, 50 μg/ml ascorbate, and 3 mM β-glycerophosphate (βGP) (differentiation media). Cyclopamine was purchased from EMD Biosciences, Inc. (La Jolla, Calif.). Primary human mesenchymal stem cells (HMSC) were purchased from Lonza (Walkersville, Md.), cultured and passaged in growth medium from StemCell Technologies (Vancouver, Canada) according to manufacturer's instructions. Osteogenic differentiation of HMSC was induced by treating the cells in DMEM low glucose containing antibiotics and 10% heat-inactivated FBS, 10-8 M dexamethasone, 10 mM βGP, and 0.2 mM ascorbate.

### Alkaline Phosphatase Activity and Von Kossa Staining

Alkaline phosphatase (ALP) activity assay on whole cell extracts (13,14), and von Kossa staining of cell monolayers for mineralization (16) were performed as previously described.

### Quantitative RT-PCR

Total RNA was extracted with the RNA isolation Trizol reagent from Ambion, Inc. (Austin, Tex.) according to the manufacturer's instructions. RNA (1 μg) was reverse-transcribed using reverse transcriptase from Bio-Rad (Hercules, Calif.) to make single stranded cDNA. Q-RT-PCR reactions were performed using iQ SYBR Green Supermix and an iCycler RT-PCR Detection System (Bio-Rad). Primer sequences for mouse genes Gli-1, Patched1 (Ptch1), bone-liver-kidney isozyme of alkaline phosphatase (ALP), bone sialoprotein (BSP), Runx2, osterix (OSX), osteocalcin (OCN) and GAPDH were used as previously described (14). Human primers sequences were: GAPDH 5′-CCT CAA GAT CAT CAG CAA TGC CTC CT (SEQ ID NO:1) and 3′-GGT CAT GAG TCC TTC CAC GAT ACC AA (SEQ ID NO:2), BSP 5′-AGA AGA GGA GGA GGA AGA AGA GG (SEQ ID NO:3) and 3′-CAG TGT TGT AGC AGA AAG TGT GG (SEQ ID NO:4), OSX 5′-GCG GCA AGA GGT TCA CTC GTT CG (SEQ ID NO:5) and 3′-CAG GTC TGC GAA ACT TCT TAG AT (SEQ ID NO:6); relative expression levels were calculated using the 2ΔΔCT method as previously described (15).

### Transient Transfection and Gli-Dependent Reporter Assay

Cells at 70% confluence in 24-well plates were transiently transfected with Gli-dependent firefly luciferase and Renilla luciferase vectors as we have previously described (17,18). FuGENE 6 transfection reagent (Roche Applied Science, Indianapolis, Ind.) was used at a ratio of 3:1 with nuclease-free water and total DNA per well did not exceed 500 ng. Luciferase activity was assessed using the Dual Luciferase Reporter Assay System (Promega Corporation, Madison, Wis.) according to manufacturer's instructions after cells were treated for 48 hours.

### Synthesis and Molecular Characterization of Oxy133

Materials were obtained from commercial suppliers and were used without further purification. Air or moisture sensitive reactions were conducted under argon atmosphere using oven-dried glassware and standard syringe/septa techniques. The reactions were monitored on silica gel TLC plates under UV light (254 nm) followed by visualization with Hanessian's staining solution. Column chromatography was performed on silica gel 60. 1H NMR spectra were measured in CDCl3. Data obtained are reported as follows in ppm from an internal standard (TMS, 0.0 ppm): chemical shift (multiplicity, integration, coupling constant in Hz.). Stepwise detailed description of the synthesis protocol and characterization of the intermediates and final products are provided in Supplemental Material.

### Animals

Thirty-eight 8-week-old male Lewis rats were purchased from Charles River Laboratories (Wilmington, Mass.) and were maintained and housed at the UCLA vivarium in accordance with regulations set forth by the UCLA Office of Protection of Research Subjects. The study was performed under a protocol approved by the UCLA Animal Research Committee (ARC). All animals were euthanized using a standard CO2 chamber 8 weeks after the spinal fusion procedure, and their spines were excised and stored in 40% ethyl alcohol.

### Surgical Procedures

Animals were pre-medicated with sustained release buprenorphine thirty minutes prior to surgery and anesthetized with 2% isoflurane administered in oxygen (1 L/min). The surgical site was shaved and disinfected with Betadine and 70% ethanol. Posterolateral intertransverse process spinal fusion at L4-L5 was performed as in prior studies (21,22). The L6 vertebral body was identified using the iliac crest as a landmark. A 4-cm longitudinal midline incision was made through the skin and subcutaneous tissue over L4-L5 down to the lumbodorsal fascia. A 2-cm longitudinal paramedial incision was then made in the paraspinal muscles bilaterally to expose the transverse processes of L4-L5, which were decorticated with a high-speed burr. The surgical site was then irrigated with sterile saline, and 5 mm×5 mm×13 mm pieces of collagen sponge (Helistat, Integra Life Sciences) containing dimethyl sulfoxide (DMSO) control, rhBMP-2, or Oxy149 were placed bilaterally, with each implant spanning the transverse processes. The implants were then covered with the overlying paraspinal muscles and the lumbodorsal fascia and skin were closed with 4-0 Prolene sutures (Ethicon, Inc., Somerville, N.J.). Animals were allowed to ambulate, eat, and drink ad libitum immediately after surgery.

### Radiographic Analysis

Posteroanterior radiographs of the lumbar spine were taken on each animal at 4, 6, and 8 weeks after surgery using a Faxitron LX60 cabinet radiography system and evaluated blindly by two independent observers employing the following standardized scale: 0, no fusion; 1, unilateral fusion; and 2, complete bilateral fusion. The scores from the observers were added together and only a score of 4 was considered as complete fusion.

### Manual Assessment of Fusion

Eight weeks after surgery, animals were euthanized and the spines were surgically removed and evaluated by two blinded independent observers for motion between levels. Nonunion was recorded if motion was observed between the facets or transverse processes on either side. Complete fusion was recorded if no motion was observed bilaterally. Spines were scored as either fused or not fused. Unanimous agreement was required to consider complete fusion.

### Micro-Computed Tomography

Each removed spine was analyzed by high resolution micro-computed tomography (micro-CT), using a SkyScan 1172 scanner (SkyScan, Belgium) with a voxel isotropic resolution of 20 μm and an X-ray energy of 55 kVp and 181 mA to further assess the fusion rate and observe the fusion mass as we have previously reported (15). Three hundred and sixty projections were acquired over an angular range of 180° with steps of 0.5 with an exposure time of 220 msec/slice. Five frames were averaged at each rotation step to get better signal to noise ratio. A 0.5 mm aluminum filter was used to narrow down the X-ray beam frequency in order to minimize beam hardening artifact. Virtual image slices were reconstructed using the cone-beam reconstruction software version 2.6 based on the Feldkamp algorithm (SkyScan). These settings produced serial cross-sectional 1024×1024 pixel images. Sample re-orientation and 2D visualization were performed using DataViewer (SkyScan). 3D visualization was performed using Dolphin Imaging version 11 (Dolphin Imaging & Management Solutions, Chatsworth, Calif.). Fusion was defined as the bilateral presence of bridging bone between the L4 and L5 transverse processes. The reconstructed images were judged to be fused or not fused by two experienced independent observers. To quantify the density of bone formed within each fusion mass, the tissue volume of the mass (TV), trabecular bone volume within the mass (BV), BV/TV ratio, trabecular thickness, and trabecular separation were calculated. This was performed using DataViewer software with measurements across 501 axial slices (20 um per slice, 10.02 mm length) within each fusion mass, centered at the level of the intervertebral body of L4-5.

### Histology

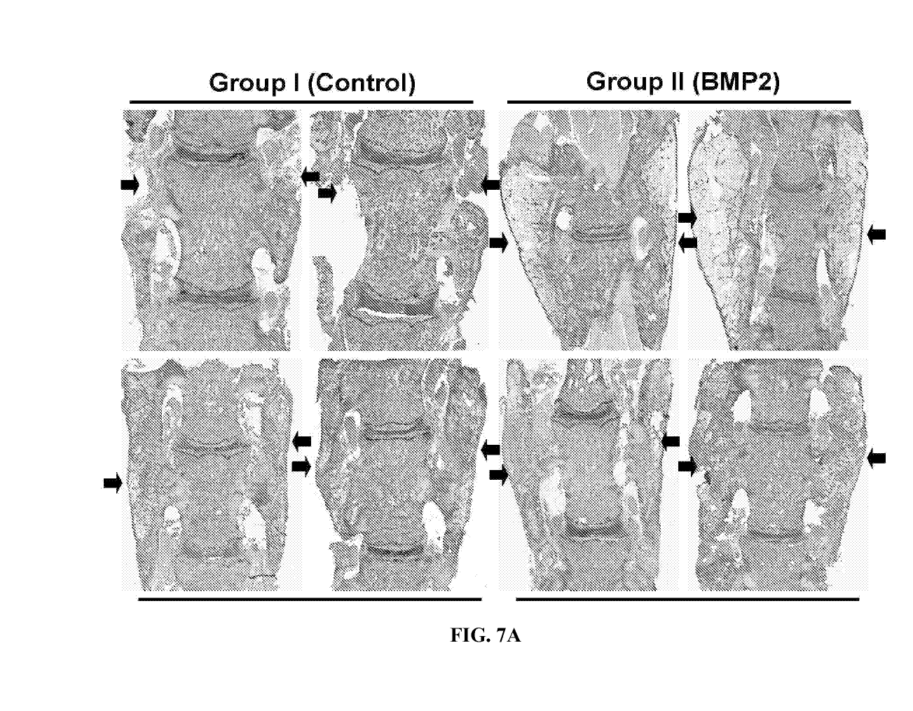

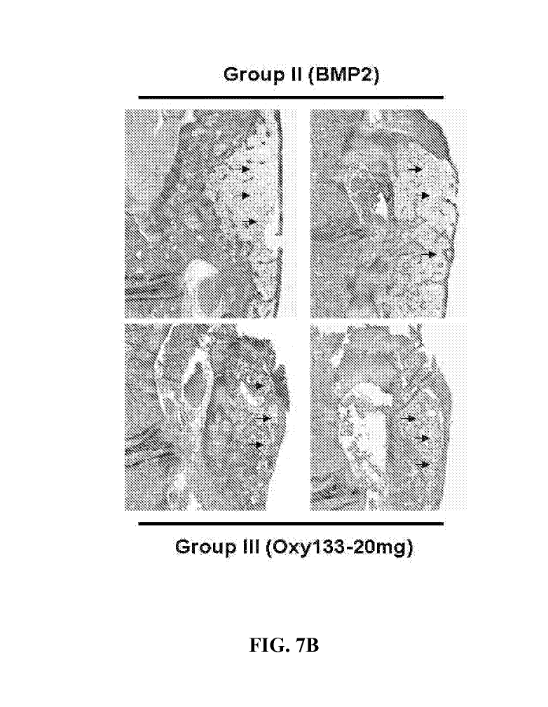

After undergoing micro-CT, two representative specimens from each surgical group were processed undecalcified by dehydration, clearing in xylene and embedding in methyl methacrylate as we have previously reported (15,23). Serial coronal sections were cut with a thickness of Sum and stained with toluidine blue pH 6.4. Photomicrographs of sections were obtained as previously reported using a ScanScope XT System (Aperio Technologies, Inc., Vista, Calif.) at a magnification of 10× in FIG. 7A and 20× in FIG. 7B (24).

### Statistical Analysis

Statistical analyses were performed using the StatView 5 program. All p values were calculated using ANOVA and Fisher's projected least significant difference (PLSD) significance test. A value of p<0.05 was considered significant.

### Example II

### Synthetic Scheme for the Synthesis of Oxy133 and its Linkage to the Bone Targeting Agent in Order to Create the Hybrid Molecule OXY149

Materials were obtained from commercial suppliers and were used without further purification. Air or moisture sensitive reactions were conducted under argon atmosphere using oven-dried glassware and standard syringe/septa techniques. The reactions were monitored on silica gel TLC plates under UV light (254 nm) followed by visualization with Hanessian's staining solution. Column chromatography was performed on silica gel 60. ¹H NMR spectra were measured in CDCl₃. Data obtained are reported as follows in ppm from an internal standard (TMS, 0.0 ppm): chemical shift (multiplicity, integration, coupling constant in Hz.). The following is a stepwise description of the protocol. Structures of Oxy34 and Oxy49, the synthesis of which some of the inventors had previously reported [Johnson et al. (2011), Journal of Cellular Biochemistry 112, 1673-1684], are shown for comparison to the structure of Oxy133.

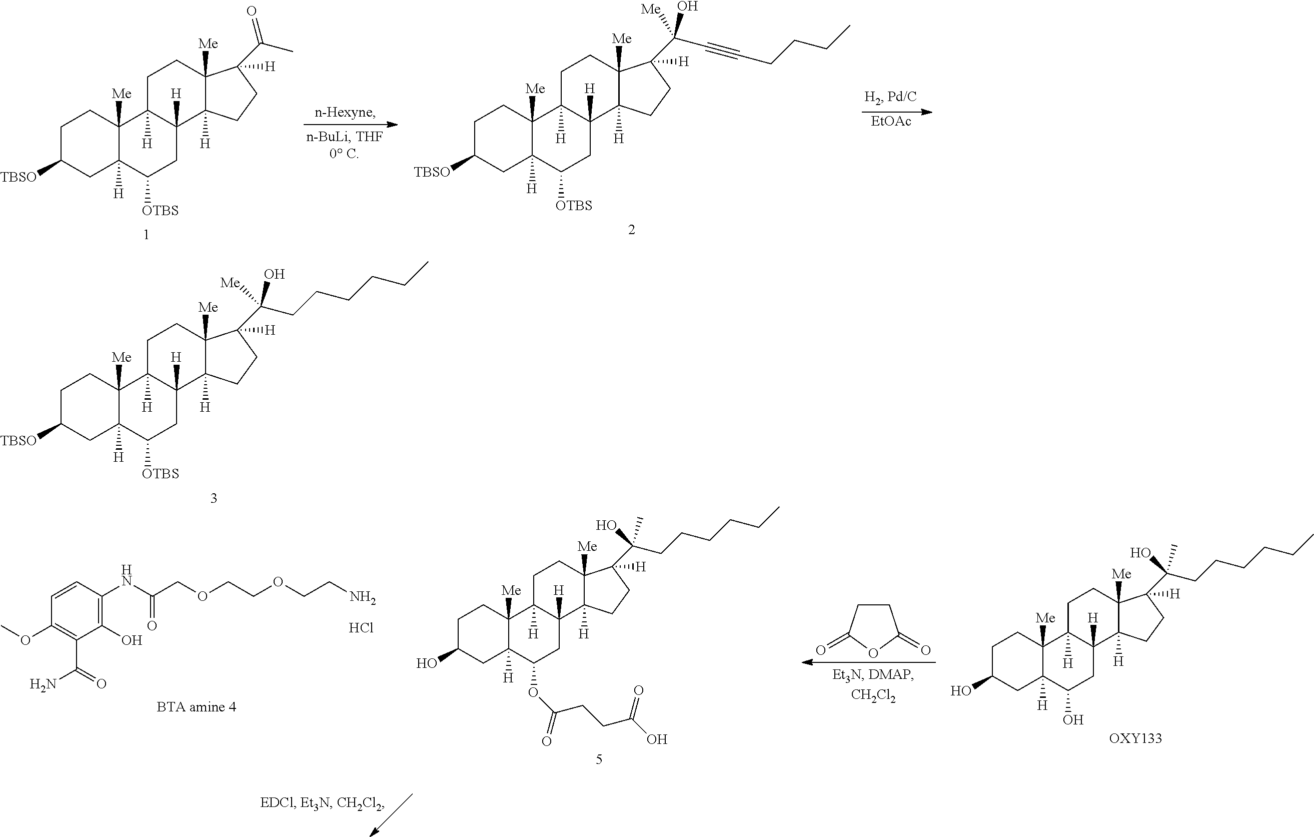

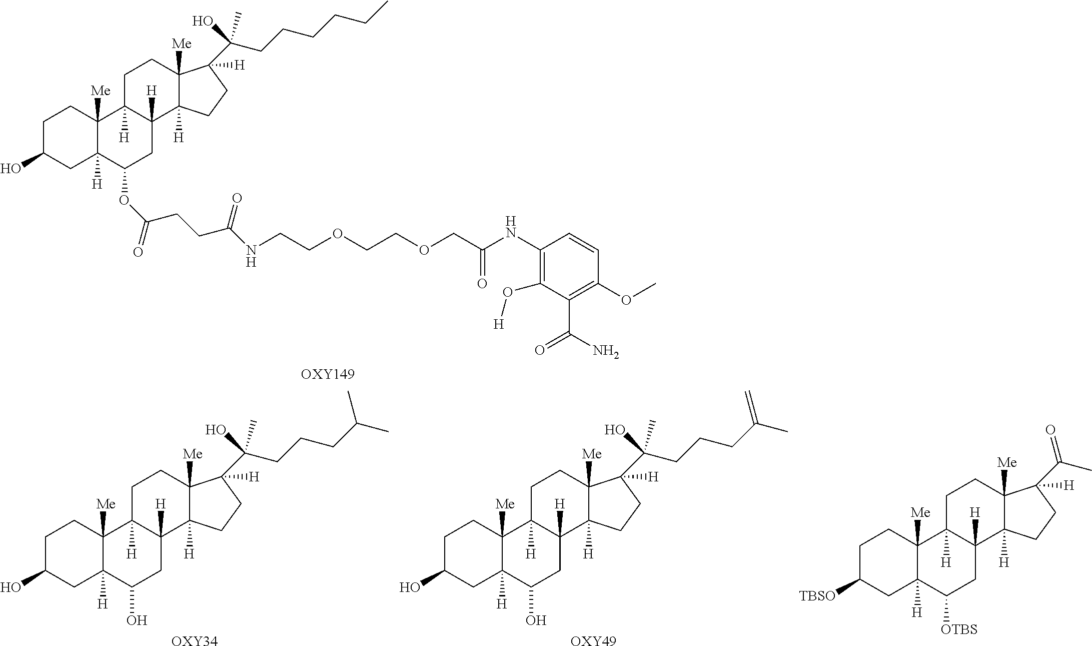

### 1-((3S,5S,6S,8R,9S,10R,13S,14S,17S)-3,6-bis((tert-butyldimethylsilyl)oxy)-10,13-dimethylhexadecahydro-1H-cyclopenta[a]phenanthren-17-yl)ethanone (1)

Prepared according to a published patent procedure [Parhami et al. (2009), WO 2009/07386, pp. 52]

¹H NMR (CDCl3, 400 MHZ) δ: 3.47 (1H, dddd, J=11.0, 11.0, 4.8, 4.8 Hz), 3.36 (1H, ddd, J=10.4, 10.4, 4.4 Hz), 2.53 (1H, d, J=8.8, 8.8 Hz), 2.20-2.14 (1H, m), 2.10 (3H, s), 2.01-1.97 (1H, m), 1.88-1.82 (1H, m), 1.73-0.89 (17H, m), 0.88, 18H, s), 0.79 (3H, s), 0.59 (3H, s), 0.043 (3H, s), 0.04 (3H, s), 0.03 (3H, s), 0.02 (3H, s). ¹³C NMR (CDCl3, 100 MHZ) δ: 209.5, 72.2, 70.1, 63.7, 56.4, 53.7, 51.8, 44.2, 41.9, 38.9, 37.6, 36.3, 34.3, 33.2, 31.7, 31.5, 25.94, 25.92, 24.4, 22.7, 21.1, 18.3, 18.1, 13.5, 13.4, −4.1, −4.6, −4.7.

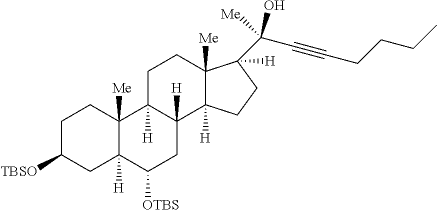

### (R)-2-((3S,5S,6S,8R,9S,10R,13S,14S,17S)-3,6-bis((tert-butyldimethylsilyl)oxy)-10,13-dimethylhexadecahydro-1H-cyclopenta[a]phenanthren-17-yl)oct-3-yn-2-ol (2)

To a cold (0° C.) solution of n-hexyne (1.5 mL, 12 mmol) in THF (6 mL) was added a 1.6 M solution of n-BuLi in hexanes (3.75 mL). The resulting solution was stirred for 30 min until a solution of compound 1 (1.27 g, 2.2 mmol) in THF (10 mL) was added via cannula. The mixture was warmed to room temperature over 3 h and diluted with water (40 mL) and the crude product was isolated by ethyl acetate extraction (3×30 mL). The combined organic layers were washed with brine and dried over Na₂SO₄. Concentration gave an oily product which was purified on silica gel (hexane, EtOAc, gradient). There was 1.30 g of product 2 (92%).

¹H NMR (CDCl3, 300 MHZ) δ: 3.50 (1H, ddd, J=15.9, 11.0, 4.8 Hz), 3.36 (1H, dt, J=10.6, 4.3 Hz), 2.18 (1H, t, t=6.9 Hz), 2.10 (1H, m), 1.91-1.62 (4H, m), 1.53-1.31 (2H, m, 3H, s), 1.31-0.93 (22H, m), 0.93 (3H, s), 0.92 (3H, m), 0.90 (18H, s), 0.88 (3H, s), 0.61 (1H, m), 0.04 (6H, s), 0.03 (6H, s). ¹³C NMR (CDCl3, 75 MHZ) δ: 85.9, 83.9, 72.4, 71.4, 70.3, 60.5, 55.8, 53.8, 51.8, 43.5, 36.3, 33.7, 33.0, 30.7, 25.9, 22.0, 18.4, 18.3, 18.1, 13.6, 13.5, −4.7, −4.7.

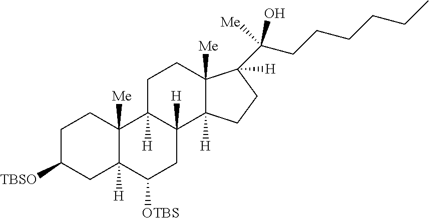

### (S)-2-((3S,5S,6S,8R,9S,10R,13S,14S,17S)-3,6-bis((tert-butyldimethylsilyl)oxy)-10,13-dimethyl hexadecahydro-1H-cyclopenta[a]phenanthren-17-yl)octan-2-ol (3)

Compound 2 (1.3 g, 2.0 mmol) was dissolved in EtOAc (5 mL), MeOH (5 mL) and Pd/C (10%, 0.1 g) was added to the solution. The mixture was degassed repeatedly under vacuum and then exposed to hydrogen gas under atmospheric pressure (balloon). After 18 h at room temperature, the mixture was diluted with EtOAc (20 mL) and filtered over Celite to remove the catalyst. The filter washed with EtOAc and the combined filtrates evaporated. There was 1.3 g of reduced product 3 which was used without further purification.

¹H NMR (CDCl3, 300 MHZ) δ:3.50 (1H, ddd, J=15.9, 11.0, 4.8 Hz), 3.36 (1H, dt, J=10.6, 4.3 Hz), 2.1-1.95 (2H, m), 1.75-1.35 (10H, m), 1.32-1.29 (10H, m, 3H, s), 0.91-1.21 (10H, m), 0.89 (18H, s), 0.82 (3H, s), 0.79 (3H, s), 0.63 (3H, m), 0.04 (6H, s), 0.03 (6H, s)¹³C NMR (CDCl3, 75 MHZ) δ: 75.2, 72.3, 57.6, 56.4, 53.8, 51.8, 42.9, 37.6, 36.3, 33.7, 31.9, 30.0, 25.9, 22.6, 18.3, 18.1, 14.1, 13.8, 13.5, −4.6, −4.7.

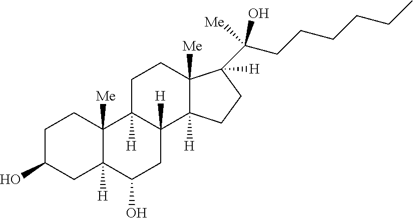

### (3S,5S,6S,8R,9S,10R,13S,14S,17S)-17-((S)-2-hydroxyoctan-2-yl)-10,13-dimethylhexadecahydro-1H-cyclopenta[a]phenanthrene-3,6-diol (OXY133)

A 1 M solution of TBAF in THF (8 mL, 8 mmol, 4 equiv) was directly added to compound 3 (1.3 g, 2.0 mmol, 1.0 equiv) and the resulting solution was diluted with THF (1 mL) and stirred at room temperature for 72 hours. The mixture was then diluted with water (50 mL) and extracted repeatedly with EtOAc (4×40 mL). The combined organic layers were washed with brine, dried over Na₂SO₄ and the solvent evaporated. Purification of the crude product by silica gel chromatography (hexane, EtOAc, gradient, then 10% MeOH in EtOAc) afforded a white solid (0.6 g, 70%) which was subjected to trituration in aqueous acetone (acetone, water, 3:1).

¹H NMR (CDCl3, 300 MHZ) δ: 3.50 (1H, ddd, J=15.9, 11.0, 4.8 Hz), 3.36 (1H, dt, J=10.6, 4.3 Hz), 2.19 (1H, m), 2.10-1.90 (3H, m), 1.85-1.60 (7H, m), 1.55-1.38 (7H, m), 1.25 (11H, brs), 1.20-0.95 (4H, m), 0.90 (3H, m), 0.86 (3H, s), 0.80 (3H, s) 0.62 (2H, m). ¹³C NMR (CDCl3, 75 MHZ) δ: 75.1, 71.1, 69.3, 57.5, 56.2, 53.6, 51.6, 44.0, 42.8, 41.4, 40.1, 37.2, 36.2, 33.5, 32.1, 31.8, 30.9, 29.9, 26.3, 24.2, 23.6, 22.5, 22.2, 20.9, 14.0, 13.6, 13.3. MS: M+H=420.36. HRMS (ESI) m/z [M−2H₂OH]⁺ calcd for C₂₇H₄₄OH: 385.3470. found 385.3478.

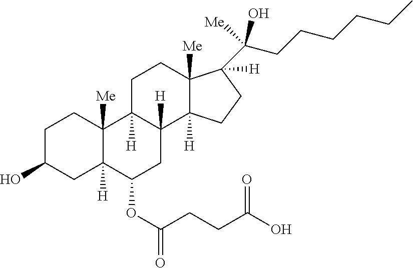

### 4-(((3S,5S,6S,8R,9S,10R,13S,14S,17S)-3-hydroxy-17-((S)-2-hydroxyoctan-2-yl)-10,13-dimethylhexadecahydro-1H-cyclopenta[a]phenanthren-6-yl)oxy)-4-oxobutanoic acid (6)

To a solution of OXY133 (80 mg, 0.2 mmol) in CH₂Cl₂ (2 mL) was added Et₃N (0.08 mL), DMAP (˜1 mg, 5 mol %) and succinic anhydride (20 mg, 1 eq). The mixture was stirred at room temperature for six hours after which a second portion of succinic anhydride was added (20 mg, 1 eq). After 18 hours at room temperature, the reaction mixture was diluted with saturated NaHCO₃ solution (20 mL) and CH₂Cl₂ (10 mL). The layers were separated and the aqueous layer extracted with CH₂Cl₂ (3×10 mL). The combined organic layers were washed with 0.5 M HCl solution and water, dried over Na₂SO₄ and the solvent evaporated. The crude product was purified by silica gel chromatography (EtOAc, then 10% MeOH in EtOAc) to afford a fraction rich in recovered staring material, a fraction rich in desired compound 6 (40 mg, 38%) and mixed fractions containing 6 and its regioisomer.

¹H NMR (CDCl3, 300 MHZ) δ: 4.71 (1H, ddd, J=15.9, 11.0, 4.8 Hz), 3.36 (1H, dt, J=10.6, 4.3 Hz), 2.65 (4H, m), 2.19 (1H, m), 2.10-1.90 (3H, m), 1.85-1.60 (7H, m), 1.55-1.38 (7H, m), 1.25 (11H, brs), 1.20-0.95 (4H, m), 0.90 (3H, m), 0.86 (3H, s), 0.80 (3H, s) 0.62 (2H, m).

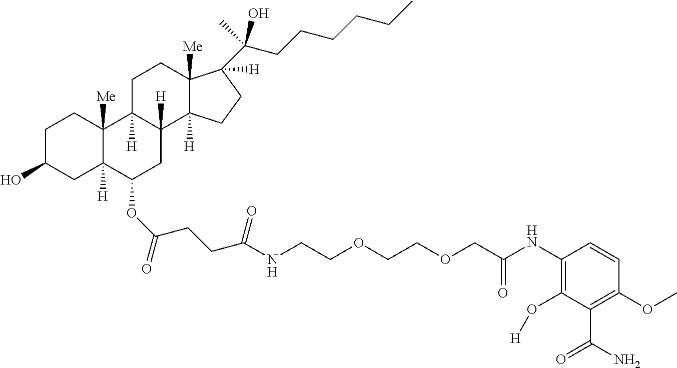

### (3S,5S,6S,8R,9S,10R,13S,14S,17S)-3-hydroxy-17-((S)-2-hydroxyoctan-2-yl)-10,13-dimethylhexadecahydro-1H-cyclopenta[a]phenanthren-6-yl 4-((2-(2-(2-((3-carbamoyl-2-hydroxy-4-methoxyphenyl)amino)-2-oxoethoxy)ethoxy)ethyl)amino)-4-oxobutanoate

### (OXY149)

To a solution of compound 6 (0.1 g, 0.19 mmol) in CH₂Cl₂ (2 mL) was added Et₃N (0.1 mL) followed by BTA amine HCl salt 4 (0.15 g, 0.41 mmol, 1.7 eq) and the mixture was stirred for 10 min. Then EDCl (140 mg, 3 eq) was added to the mixture in one portion. The mixture was stirred at room temperature for 72 h under inert atmosphere (forming a viscous syrup as the solvent partially evaporated). Then, the reaction mixture was diluted with saturated NaHCO₃ solution (20 mL) and CH₂Cl₂ (10 mL). The layers were separated and the aqueous layer extracted with CH₂Cl₂ (3×10 mL). The combined organic layers were washed with 0.5 M HCl solution and water, dried over Na₂SO₄ and the solvent evaporated. The crude product was purified twice by silica gel chromatography (first 10% MeOH in EtOAc, then in CH₂Cl₂, MeOH 1-3%) to afford OXY149 (50 mg, 32%) with an estimated purity of 90%. ¹H NMR (CDCl3, 300 MHZ) δ: 14.62 (1H, m), 8.39 (1H, d, j=9 Hz), 6.40 (3H, d, J=9 Hz), 4.73 (1H, m), 4.14 (2H, s), 3.92 (3H, s), 3.76-3.34 (9H, m), 2.57-2.42 (4H, m), 2.19 (1H, m), 2.10-1.90 (3H, m), 1.85-1.60 (8H, m), 1.55-1.38 (8H, m), 1.25 (12H, brs), 1.20-0.95 (4H, m), 0.90 (3H, m), 0.86 (3H, s), 0.80 (3H, s) 0.62 (2H, m). ¹³C NMR (CDCl3, 75 MHZ) δ: 172.5, 172.4, 171.7, 168.2, 154.7, 154.3, 124.2, 121.1, 102.7, 100.2, 75.1, 73.9, 71.3, 70.3, 70.1, 69.4, 69.3, 57.7, 56.3, 53.7, 51.7, 51.6, 44.1, 42.9, 41.4, 40.1, 39.3, 37.0, 36.3, 33.6, 32.3, 31.9, 31.1, 30.0, 29.7, 28.3, 27.1, 26.4, 24.3, 23.7, 22.7, 21.0, 14.1, 13.7, 13.4. Analytical HPLC: Phenomenex C-18 column (Gemini, 3×100 mm, 5 microns). A: water, formic acid (999:1), B: Acetonitrile, formic acid (999:1). % B after each minute of run time: 0, 0.5, 8, 10, 20, 20, 100, 100. Retention time 8.1 min, MS: M+H=831.4.

### Example III

### Experimental Results: Stimulation by Oxy133 of Osteogenesis of Bone Formation and Spinal Fusion In Vivo

### Oxy133 Induces Osteogenic Differentiation of Bone Marrow Stromal Cells, Embryonic Fibroblasts, and Human Mesenchymal Stem Cells

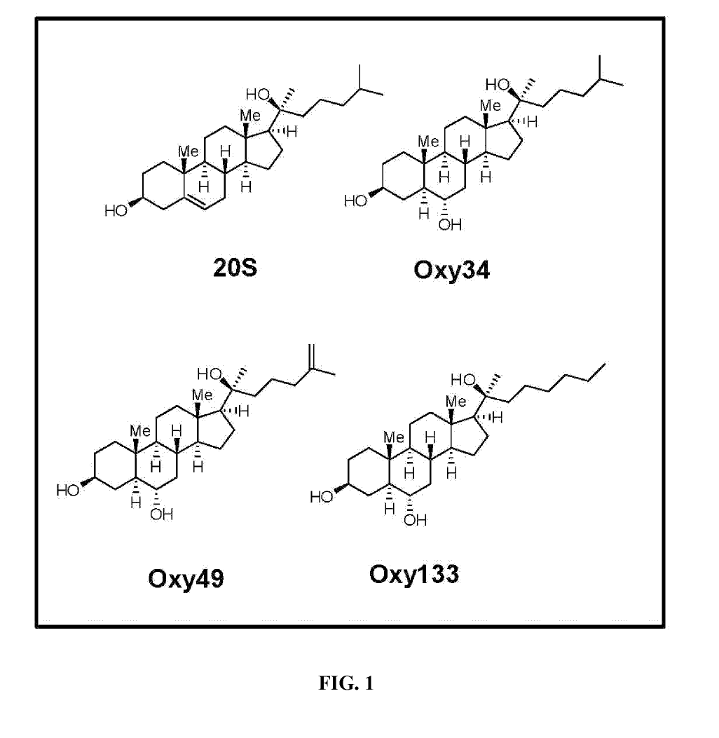

To achieve the goal of developing a molecule capable of inducing osteogenic differentiation of osteoprogenitor cells, we modified the molecular structure of the most potent osteogenic naturally occurring oxysterol, 20(S)-hydroxycholesterol (20S) based on our understanding of the structure activity relationships observed in over 100 previously synthesized analogues. We previously reported that robust osteogenic differentiation was achieved with two structural analogues of 20S, Oxy34 and Oxy49 (15). These molecules were formed by adding an a hydroxyl (OH) group on carbon 6 (C6) in both Oxy34 and 49, and a double bond between C25 and C27 in Oxy49 (FIG. 1) (15). In studies reported here, we attempted to further improve on these two molecules by developing a more easily synthesized and more potent analogue that would be suitable for scale up into large amounts for future preclinical and clinical studies in large animals and humans, respectively. This molecule could be a candidate for therapeutic development and clinical use to increase bone formation locally for stimulation of spinal fusion and fracture healing, and perhaps even systemically to address disorders such as osteopenia and osteoporosis. Through structure activity relationship studies a novel analogue, Oxy133, was synthesized according to the protocol described in Example II and tested for osteoinductive activity. Oxy133 differs from Oxy34 and 49 by the deletion of C27 and increasing the length of the side chain by one carbon (FIG. 1). Importantly, Oxy133 can be more readily prepared on large scale due to inexpensive commercially available starting materials that result in a significantly less costly product compared to Oxy34 and Oxy49. Moreover, the alkyne addition used in the preparation of Oxy133 is superior to the Grignard chemistry used in the synthesis of Oxy34 and Oxy49 in terms of yield, purity of products (diastereoselectivity), and scalability.

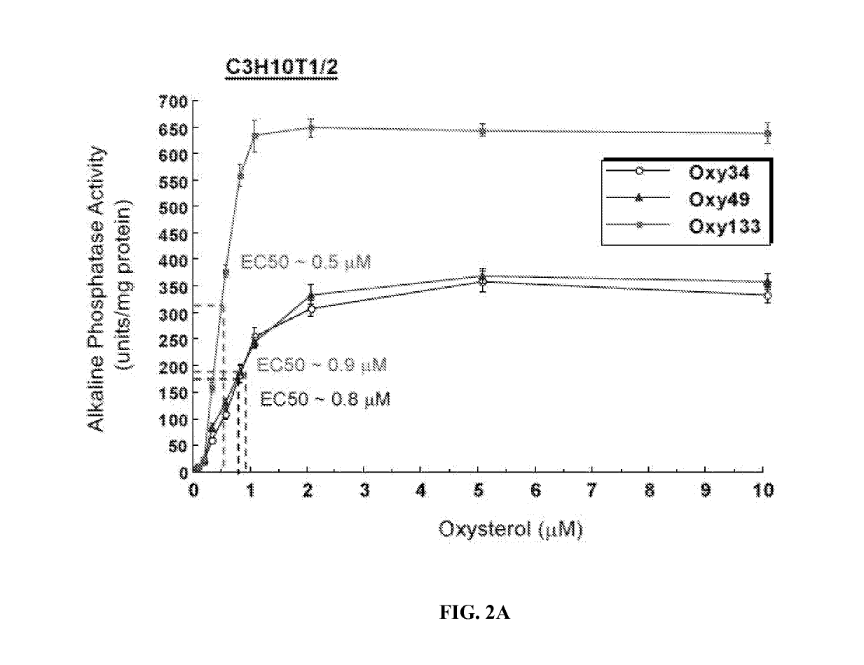

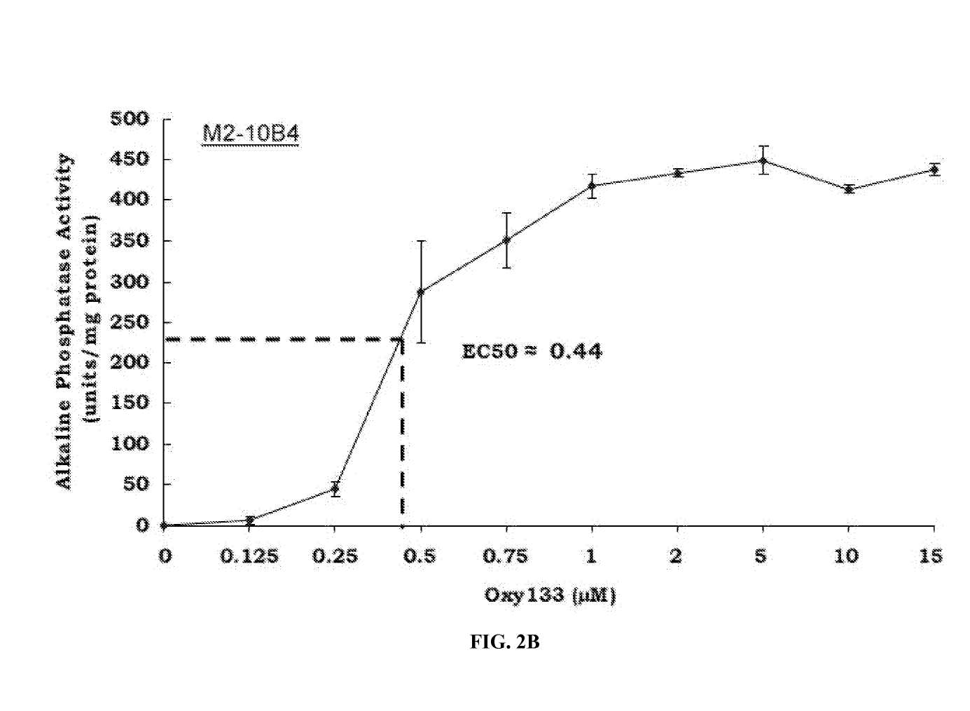

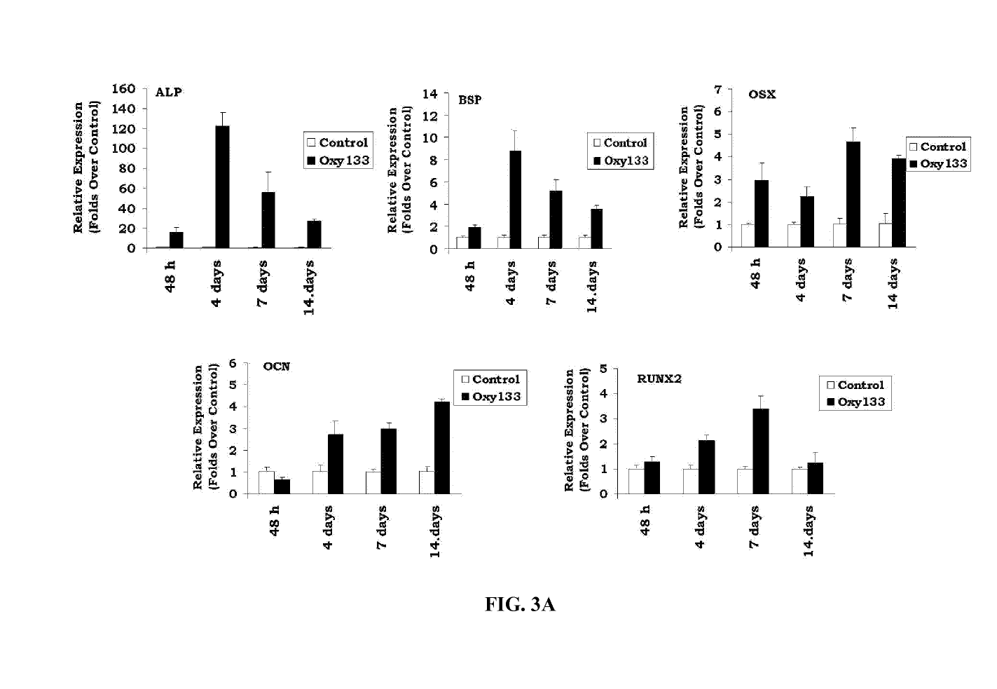

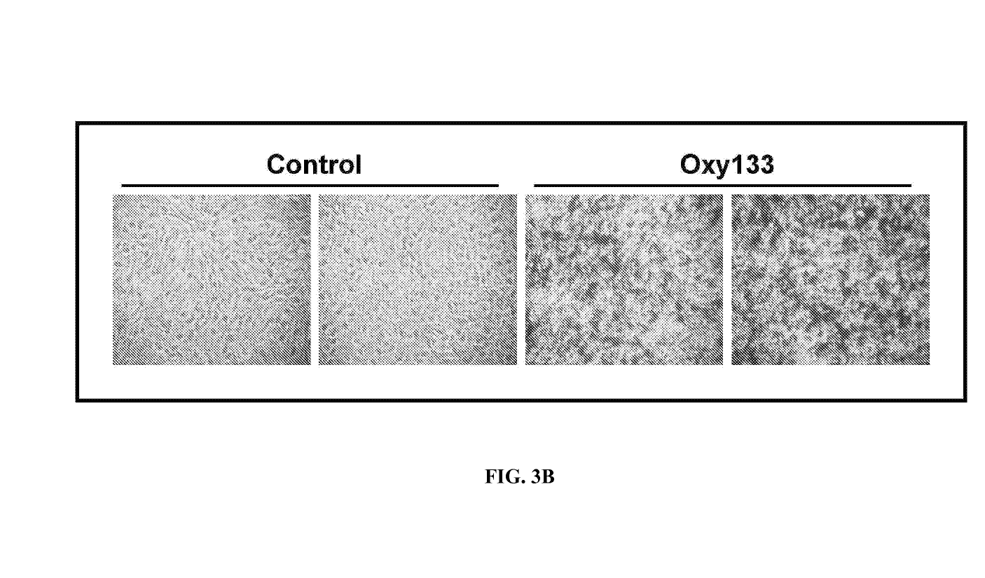

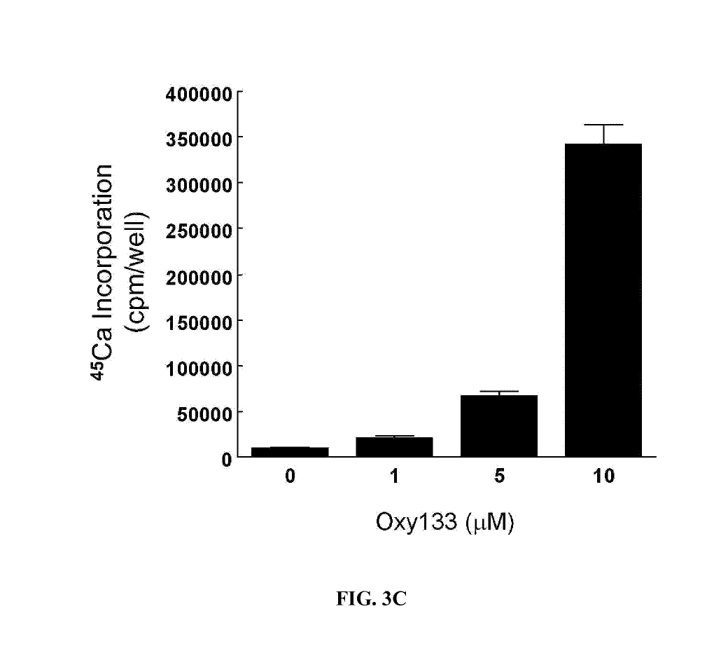

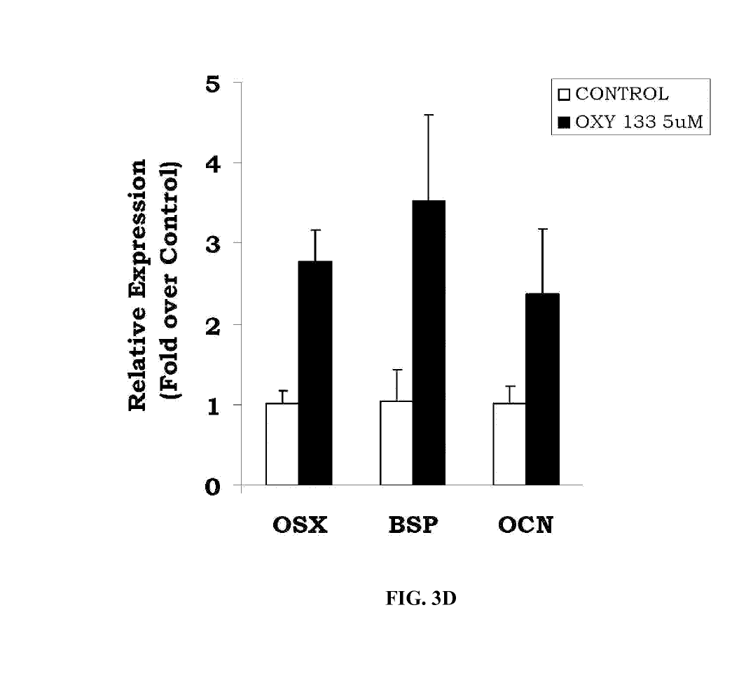

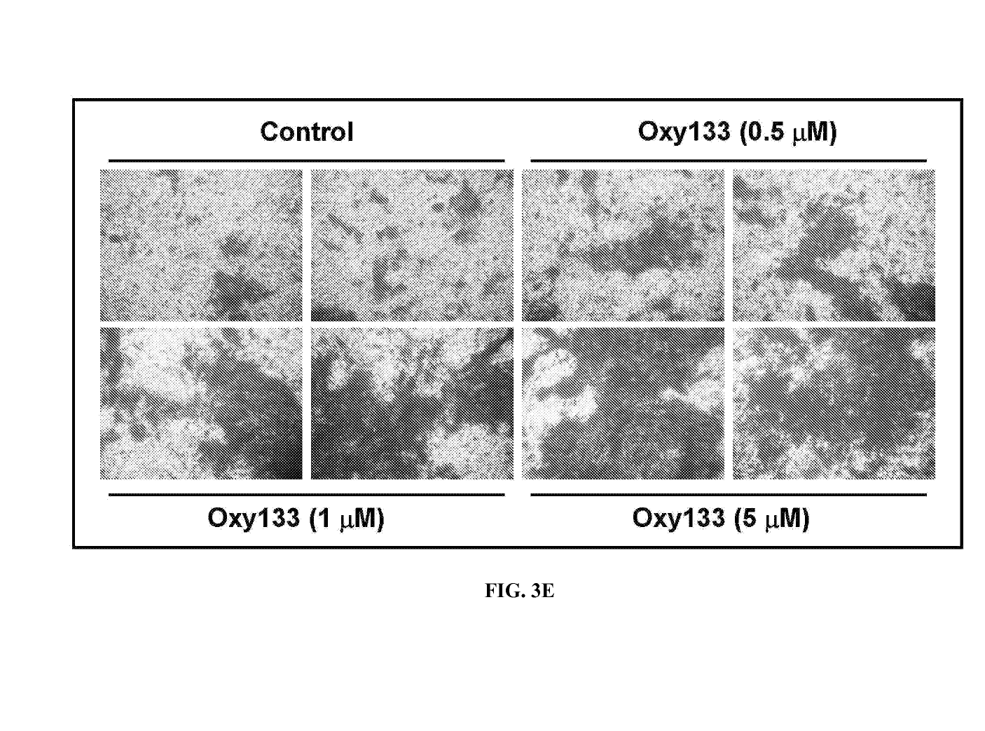

As compared with other structural analogues of 20S, Oxy133 has surprisingly improved potency in inducing alkaline phosphatase (ALP) activity as measured by ALP enzymatic activity assay in C3H and M2 cells. This is a useful model for osteogenic activity, as we have previously reported for other oxysterol analogues (15). A dose-dependent increase in ALP activity was observed with Oxy133 at low micromolar (μM) concentrations (FIG. 2A, B). The EC50 for Oxy133 was found to be approximately 0.5 μM in C3H (FIG. 2A) and 0.44 μM in M2 cells (FIG. 2B). The EC50 of Oxy34 and Oxy49 in C3H cells was found to be similar to what was previously reported in M2 cells, 0.8 and 0.9 μM, respectively, and significantly higher than the EC50 of Oxy133 (FIG. 2A). Moreover, Oxy133 at high doses induced a greater level of ALP activity than similar doses of Oxy34 and Oxy49 in C3H cells (FIG. 2A). Oxy133 was found to have other beneficial effects in inducing osteogenic differentiation of cells through analysis of the expression of osteogenic differentiation marker genes Runx2, Osterix (OSX), ALP, bone sialoprotein (BSP), and osteocalcin (OCN). In C3H cells treatment with 2.5 μM Oxy133 induced Runx2 expression 2 and 3.2 fold after 4 and 7 days of treatment, respectively, which returned to baseline levels at 14 days (FIG. 3A). OSX expression was significantly induced 3 fold after 2 days and remained elevated throughout the experiment reaching a maximum induction of 4.5 fold (FIG. 3A). Treatment of C3H cells with Oxy133 induced the expression of ALP 18 fold after 2 days which maximized to 120 fold after 4 days and then dropped to 22 fold after 7 and 14 days, respectively (FIG. 3A). BSP expression was maximally induced 9 fold on day 4 and remained induced for the duration of the experiment in spite of lowering with the longer exposure of cells to Oxy133 (FIG. 3A). Oxy133 treatment also induced the expression of osteoblast-specific gene, osteocalcin, 2.8 fold after 4 days and reached a maximum of 4.2 fold after 14 days post-treatment (FIG. 3A). Oxy133 induced robust matrix mineralization in cultures of C3H cells as determined by von Kossa staining (FIG. 3B) and quantitative extracellular matrix 45Ca assay after 21 days of treatment (FIG. 3C). These data demonstrate the efficacy and potency of Oxy133 as an osteoinductive oxysterol.

The osteogenic effects of Oxy133 were also examined in primary human mesenchymal stem cells (MSC) by assessing the expression of osteogenic genes 1 week, 2 weeks and 4 weeks post-treatment. ALP expression was high in untreated cells at all time points and there was no change with Oxy133 treatment (data not shown). After one week, a significant 2 fold increase in BSP expression was observed that was further increased to 4 fold after 2 and 4 weeks (FIG. 3D). Oxy133 also induced a significant induction of OSX (3 fold) and OCN (2 fold) after 4 weeks (FIG. 3D). Additionally, Oxy133 stimulated robust extracellular matrix mineralization in cultures of primary human MSC cells as demonstrated by von Kossa staining after 5 weeks of treatment (FIG. 3E).

### Oxy133 Induces Osteogenic Differentiation Through Activation of Hedgehog Pathway Signaling

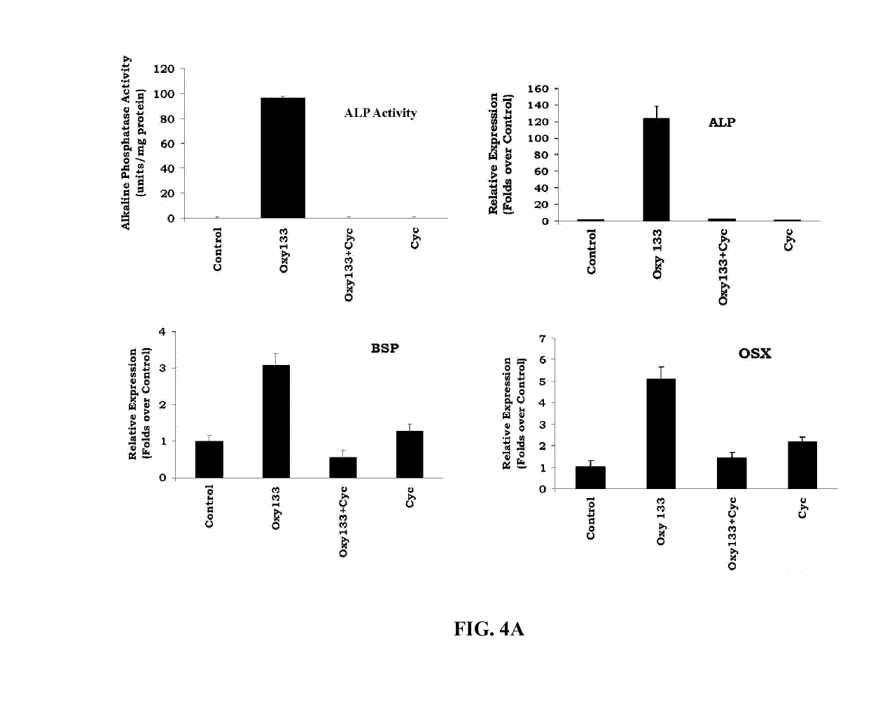

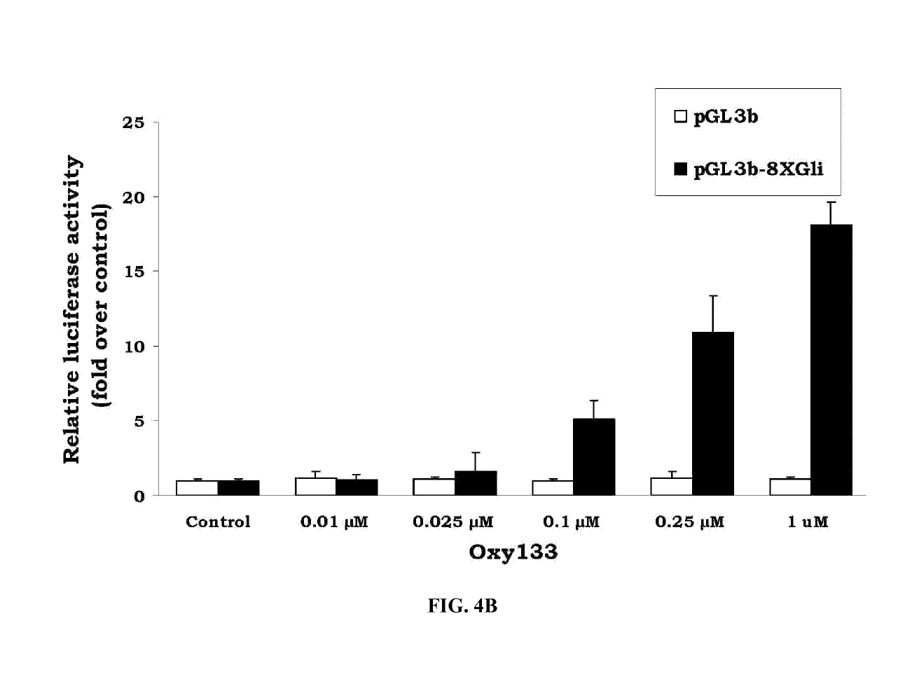

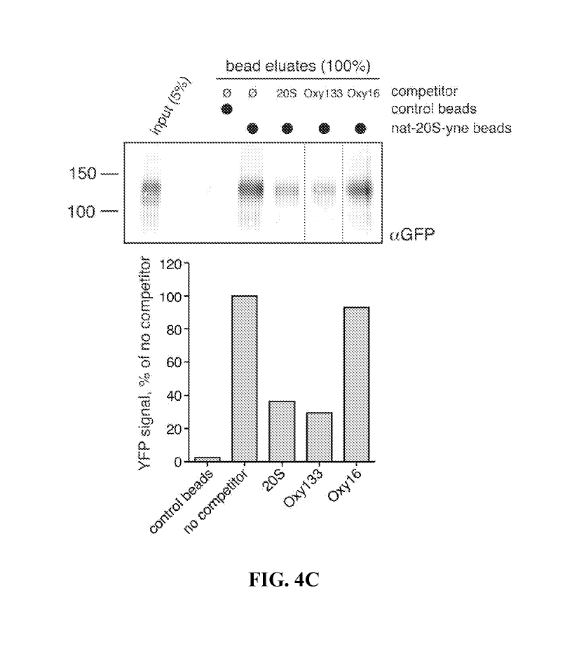

Prior research has demonstrated that 20S and its structural analogues Oxy34 and Oxy49 induce osteogenic differentiation via activation of Hh pathway signaling (15). However, the molecular mechanism for osteogenic oxysterol-mediated activation of Hh pathway signaling was not previously known. Given its greater osteogenic activity, Oxy133 is a useful tool for identifying the molecular mechanism by which Hh pathway activation and osteogenesis are achieved by the semi-synthetic oxysterols. In order to determine whether and how Oxy133 induces osteogenic differentiation through the Hh pathway, the effect of the selective Hh pathway inhibitor, cyclopamine, on Oxy133-induced ALP activity and expression of osteogenic differentiation markers ALP, BSP, and OSX was examined. Cyclopamine completely inhibited Oxy133-induced ALP activity and expression of osteogenic markers ALP, BSP, and OSX, in C3H cells (FIG. 4A), as well as in M2 cells (data not shown) suggesting that Oxy133 does act via the Hh signaling pathway. To further analyze the activation of Hh signaling by Oxy133, activation of a Gli-dependent luciferase reporter transfected into C3H cells was examined using previously reported methods (15,17). Oxy133 induced a dose-dependent increase in activity of the Gli-dependent reporter, reaching a 5 fold induction at 100 nM and a 17 fold induction at 1 μM Oxy133 (FIG. 4B).

### Oxy133 Activates the Hedgehog Signaling Pathway by Binding to the Smoothened Receptor

We previously reported that 20S selectively activates Hh signaling by binding to the Smo receptor (19). To determine whether Oxy133 activates Hh signaling by the same mechanism, we tested the ability of Oxy133 to compete for YFP-tagged Smo (YFP-Smo) binding with a 20S analogue coupled to magnetic beads. As we previously reported, this analogue, nat-20S-yne, contains an alkyne moiety on the iso-octyl chain, allowing for click chemistry-mediated coupling to magnetic beads (20S-beads) (19). Using these beads for sterol-binding assays, the amount of YFP-Smo remaining on the beads relative to a no-competitor sample is measured by Western blotting. Compounds that bind Smo at the same site as 20S compete with the 20S-beads and reduce the amount of protein in the eluate. We have tested many other sterols both in Smo binding assays and Hh signaling assays and in all cases binding to Smo correlated with a change in Hh pathway activity (19). Both Oxy133 and 20S, the positive control, reduced the amount of YFP-Smo captured on 20S-coupled beads (FIG. 4C). In an important control, a structurally related analogue, Oxy16, which cannot activate Hh signaling or osteogenesis (Parhami et al. unpublished observations) failed to prevent the interaction between YFP-Smo and 20S-beads (FIG. 4C). This reduction in the amount of YFP-Smo captured by 20S-beads in the presence of free Oxy133 suggests that Oxy133 binds to the same site on Smo as 20S. It is important to emphasize that our assay is semi-quantitative and cannot be used to derive Kd for the interaction, principally because we do not know the concentration of YFP-Smo in the extract and the amount of 20S productively immobilized on beads.

### Oxy133 Stimulates Bone Formation and Spinal Fusion In Vivo

Eight week old Lewis rats were divided into five treatment groups that differed only by the reagent contained within the collagen sponge at the surgery site: Group 1-control vehicle (DMSO) only (n=7), Group 11-5 μg rhBMP-2 (n=8), Group III-20 mg Oxy133 (n=7), Group IV-2 mg Oxy133 (n=8), and Group V-0.2 mg Oxy133 (n=8). Bone formation and spinal fusion were assessed at various time points post-operatively through radiographic analysis, and at sacrifice using manual assessment, microcomputed tomography, and histology. Fusion rates at sacrifice are summarized in Table 1.

TABLE 1
Fusion Rates (%) Assessed with
Plain Radiographs, Micro-CT, and Manual Palpation

| | X-ray | Micro-CT | Manual Palpation |
|---|---|---|---|
| Control | 0 | 0 | 0 |
| BMP2 | 100 | 100 | 100 |
| Oxy133 20 mg | 100 | 100 | 86 |
| Oxy133 2 mg | 50 | 50 | 50 |
| Oxy133 0.2 mg | 0 | 0 | 0 |

### Radiographic Analysis

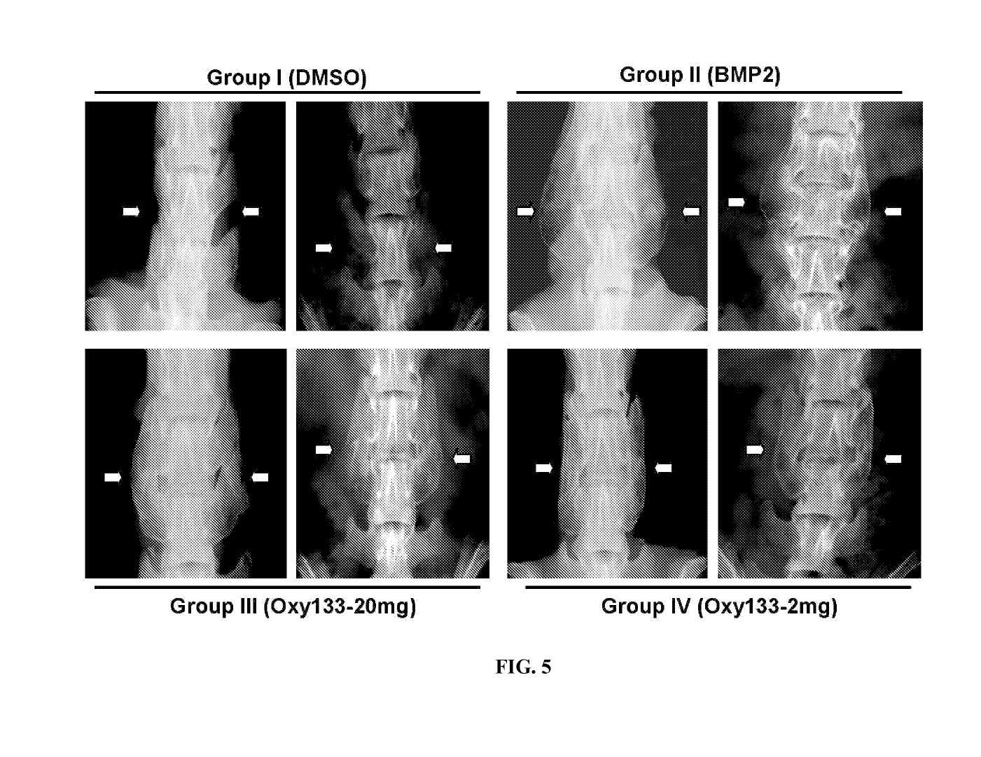

The first sets of radiographs were performed four weeks after the operation. At this time point, bilateral fusion was observed in 8/8 animals in the BMP2 group, 6/7 animals in the Oxy133-20 mg group, 3/8 animals in the Oxy133-2 mg group, and no fusion in the control and the Oxy133-0.2 mg groups. Unilateral fusion was observed in the remaining Oxy133-20 mg treated animal and in three animals treated with Oxy133-2 mg. This is in contrast to prior studies with Oxy34 and 49 in which no fusion was observed at the 4 week time point (15). By six weeks, all animals had fused bilaterally in the Oxy133-20 mg group. At eight weeks, fusion was again noted in all animals in the BMP2 and Oxy133-20 mg groups and in 4/8 of the Oxy133-2 mg group (FIG. 5). No fusion mass was observed in the DMSO or Oxy133-0.2 mg (data not shown) groups in the final eight week radiographs (FIG. 5).

### Manual Assessment and Gross Evaluation of Bone Formation

After sacrifice, the spines were explanted from each animal and subjected to manual assessment as we have previously described (15,25-27). Gross evaluation and manual assessment results were similar to radiographic findings at 8 weeks. No unilateral or bilateral fusion was observed in the DMSO or Oxy133-0.2 mg groups. Some bone formation was noted in two animals in the Oxy133-0.2 mg group. Bilateral fusion was observed in all animals in the BMP2 group and 6/7 animals in the Oxy133-20 mg group. The remaining animal in the Oxy 133-20 mg group had motion unilaterally despite significant bilateral fusion mass. Half (4/8) of the animals in the Oxy133-2 mg group had bilateral fusion confirmed on manual palpation while two additional animals had unilateral fusion and two animals had no evidence of fusion.

### Micro-Computed Tomography and Histological Assessment

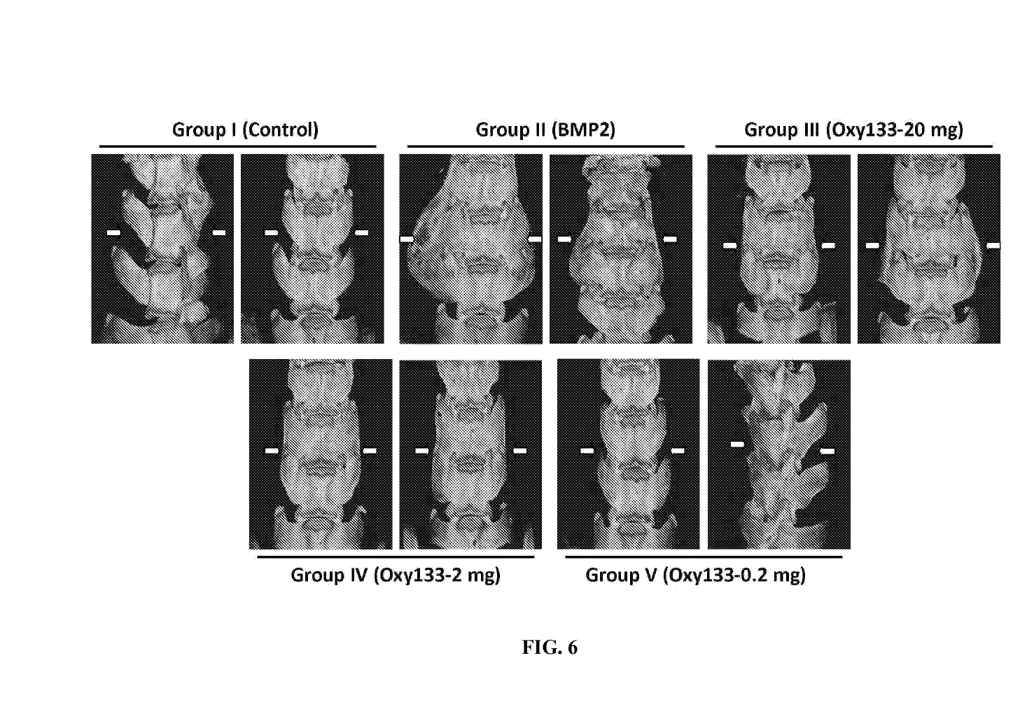

Assessment of bridging trabecular bone with micro-CT analysis confirmed results observed with radiographs, gross observation, and manual palpation (FIG. 6). Although some bone formation was seen in two animals in the Oxy133-0.2 mg group, no bilateral fusions were observed in this group or the DMSO group. Bilateral bridging trabecular bone was seen in all animals in the BMP2 group and the Oxy133-20 mg group. Bilateral fusion was also observed in 4/8 animals in the Oxy133-2 mg group with unilateral fusion in two additional animals. The results of the microstructural analysis from the micro-CT images are shown in Table 2. The total volume of the BMP2 fusion masses was significantly greater than both the Oxy133-2 mg and 20-mg samples. However, the mean BV/TV ratio of the Oxy133-2 mg and 20-mg fusion masses was significantly greater than the BMP2 group, indicating denser bone within the masses. Trabecular thickness did not significantly differ between BMP2 and either Oxy133-2 mg or Oxy133-20 mg. Trabecular separation was significantly larger in the BMP2 fusion masses compared to Oxy133-2 mg and Oxy133-20 mg, also indicating less density of bone in the BMP2 fusion masses.

TABLE 2
Quantitative Assessment of Bone
Microstructure from Micro-CT Imaging

| | Fusion | Fusion | Fusion | | Fusion |
|---|---|---|---|---|---|
| | Mass Total | Mass | Mass Bone | | Mass |
| | Tissue | Bone | Volume to | | Trabecular |
| | Volume | Volume | Tissue | | Separation |
| | (mm³) | (mm³) | Volume (%) | | (μm) |
| BMP2 5 μg | 106.909* | 20.126 | 19.633* | 131.131 | 446.126* |
| Oxy133 2 mg | 78.586 | 21.217 | 27.104 | 134.008 | 321.693 |
| Oxy133 | 79.934 | 19.592 | 24.565 | 124.737 | 310.912 |
| 20 mg | | | | | |

*indicates statistically significant difference (p < 0.01) in total tissue volume, bone volume to tissue volume ratio, and trabecular separation between BMP2 and Oxy133 20 mg and 2 mg. No differences were observed in bone volume or trabecular thickness.

Histologic analysis was then performed in two representative animals in the DMSO group, BMP2 group, Oxy133-20 mg group, and Oxy133-2 mg group. Histological assessment demonstrated the formation of trabecular bone within the fusion mass and continuous cortical bone connecting the transverse processes of the fully fused lumbar vertebrae in rats treated with BMP2, or with the 2 or 20 mg dose of Oxy133 (FIG. 7A). Bone formation was not present in specimens from control rats. The size of the fusion mass was increased in rats treated with BMP2 compared to 20 mg or 2 mg of Oxy133. However, visual inspection of the histological specimens indicated that BMP2 also induced robust formation of adipocytes within the fusion mass, which was significantly less in groups treated with Oxy133 (FIG. 7B). In addition, visual inspection suggested that trabecular bone formation was more robust in the Oxy133-20 mg group compared with BMP2 group.

### Example IV

### Studies Showing Oxy149 Activities Compared to Oxy133 Activities

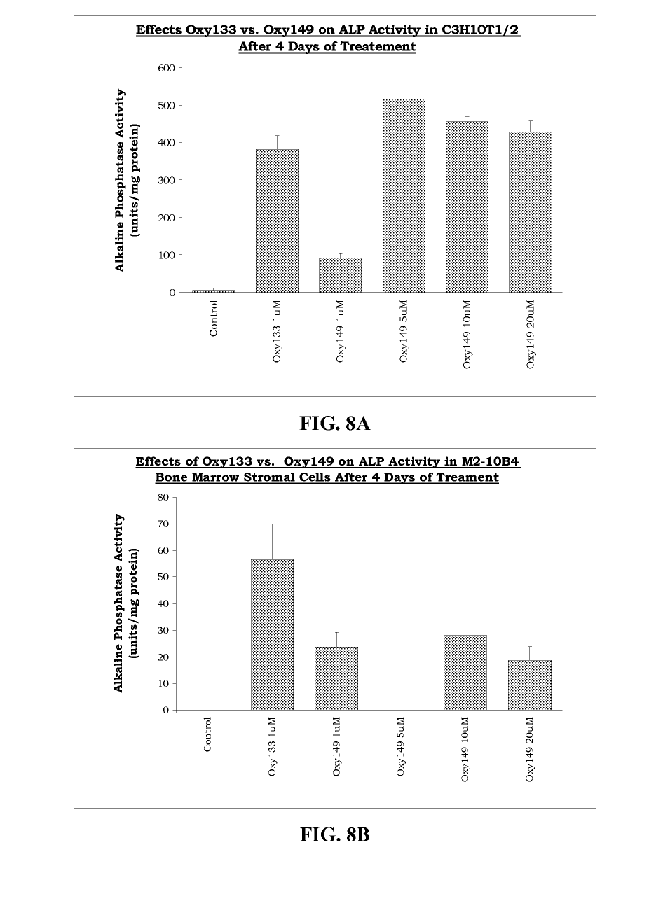

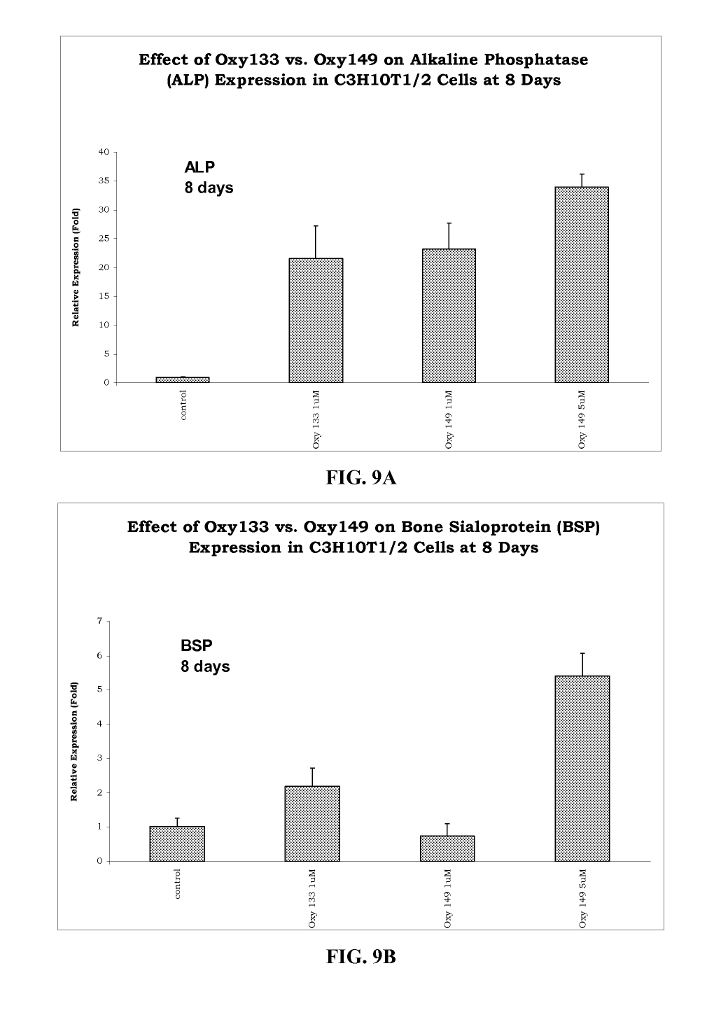

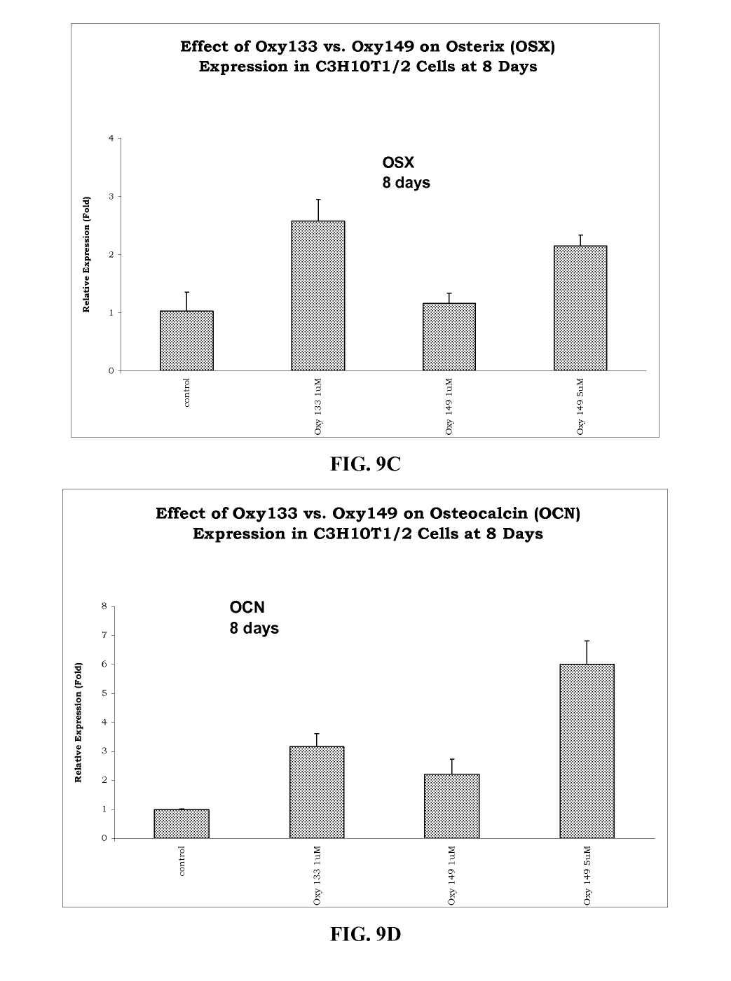

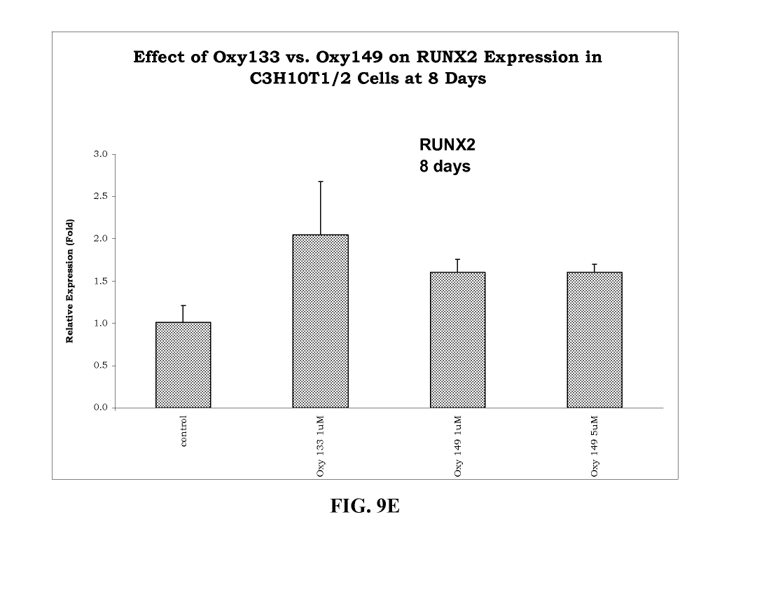

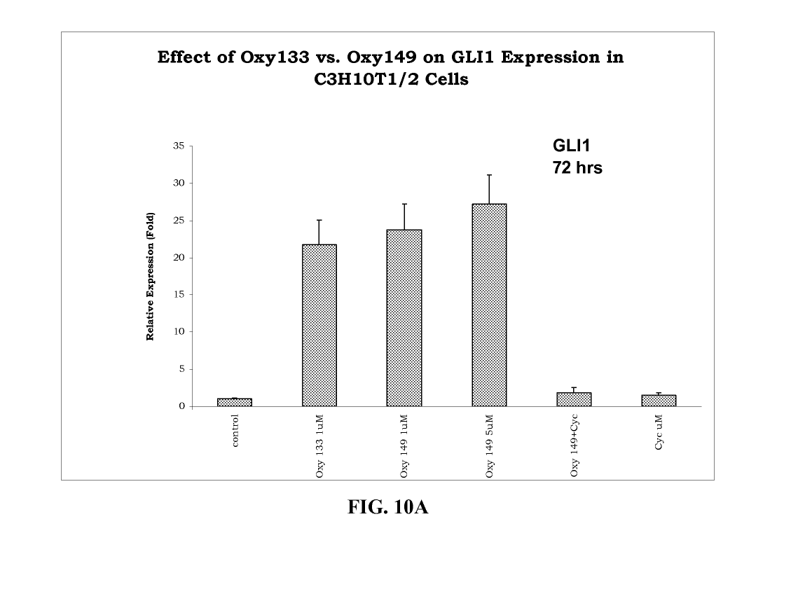

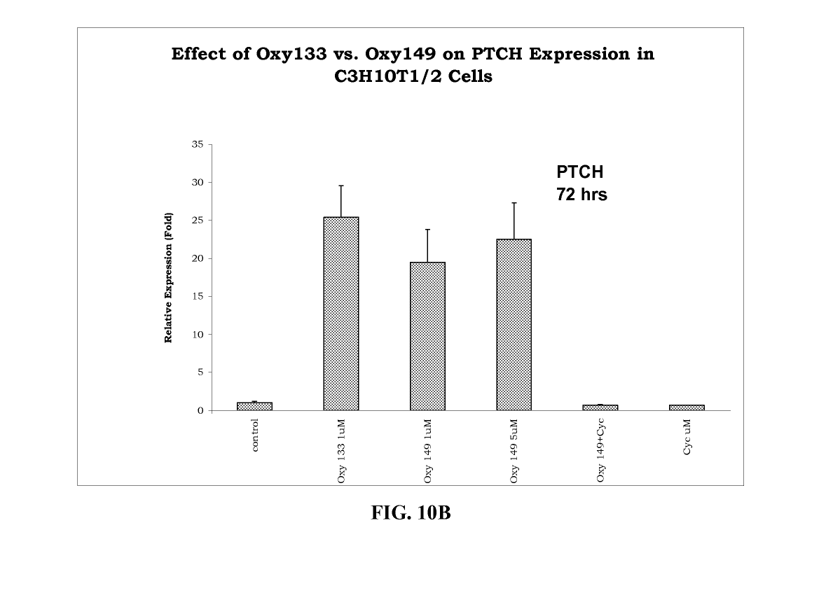

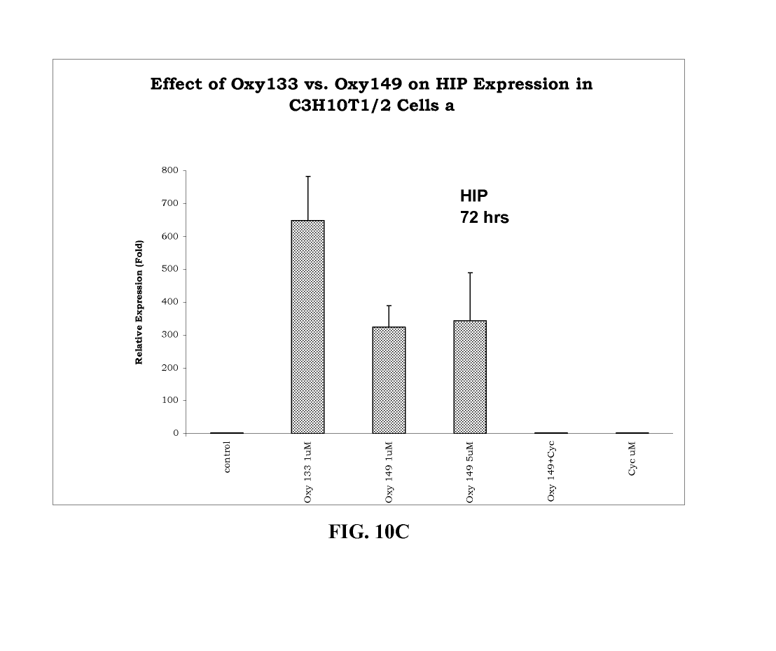

Oxy149 was tested as described above for Oxy133, and was found to stimulate osteogenic differentiation of cells in vitro. The data are shown in FIGS. 8-10, and some details of the experiments are summarized in the Description of the Drawings.

Additional experiments will also be performed with Oxy149, in vitro and in vivo, as described herein for Oxy133. It is expected that Oxy149 will display desirable potency and biological effects, e.g. when administered to a cell, tissue or organ of interest.

### Example V

### Oxy149 Effects Following Systemic Administration

Oxy149 is tested, using conventional procedures, for its beneficial properties following systemic administration to animal models. Oxy149 is examined for its ability to prevent or reverse osteoporosis in animal models of osteoporosis. Such animal models include, but are not limited to, ovariectomized mice and rats, glucocorticoid- or other drug-induced osteoporosis in rodents, and osteoporosis that results with aging in rodents and non-human primates. In these studies, Oxy149 is administered systemically through subcu, i.v., i.p., or oral administration, or through administration of a vaporized preparation of Oxy149 through nasal passages. Improvements upon treatment with Oxy149 vs. a placebo or an anti-resorptive drug will be assessed by measuring factors in the blood that change with induction of bone formation (e.g. alkaline phosphatase and osteocalcin), reduction of bone resorption (e.g. C- and N-telopeptides of collagen I), and by measuring bone density, bone mineral content, and other bone parameters using radiographs of CT imaging that determine improvements in bone microarchitecture. It is expected that because of the Oxy149's bone targeting properties, it will selectively accumulate in bone and, e.g., stimulate mesenchymal stem cells to undergo osteogenic differentiation and make new bone. Oxy149 is effective for healing fractures and preventing and/or treating osteoporosis due to its stimulation of bone formation when administered systemically to a subject

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. Provisional application 61/643,746, filed May 7, 2012, are hereby incorporated by reference in their entirety, particularly with regard to the disclosure for which they are cited in the application. Also incorporated by reference in their entirely are other applications concerning oxysterols from the present inventor's laboratory, including Patent Cooperation Treaty (PCT) international applications published as WO/2008/115469, WO/2008/082520, WO/2007/098281, WO/2007/028101, WO/2006/110490, WO/2005/020928, WO/2004/019884, and a PCT application filed on the same day as the present application, having attorney docket number 58086-342052, and based on U.S. provisional application 61/643,746, which was filed May 7, 2012.

### REFERENCES

1. Johnson E E, Urist M R 2000 Human bone morphogenetic protein allografting for reconstruction of femoral nonunion. Clin Orthop Relat Res 371:61-74.
2. Mundy G R 2002 Directions of drug discovery in osteoporosis. Annu Rev Med 53:337-54.
3. Rodan G A, Martin T J 2000 Therapeutic Approaches to Bone Diseases. Science 289:1508-14.
4. Yoon S T, Boden S D 2002 Osteoinductive molecules in orthopaedics: basic science and preclinical studies. Clin Orthop Relat Res 395:33-43.
5. Arrington E D, Smith W J, Chambers H G, Bucknell A L, Davino N A 1996 Complications of iliac crest bone graft harvesting. Clin Orthop Relat Res 329:300-9.
6. Vaccaro A R, Chiba K, Heller J G, Patel T C, Thalgott J S, Truumees E, Fischgrund J S, Craig M R, Berta S C, Wang J C 2002 Bone grafting alternatives in spinal surgery. Spine J 2:206-15.
7. Rihn J A, Kirkpatrick K, Albert T J 2010 Graft options in posterolateral and posterior interbody lumbar fusion. Spine 35:1629-39.
8. Mitka M 2011 Questions about spine fusion product prompt a new process for reviewing data. JAMA 306:1311-2.
9. Lewandrowski K-U, Nanson C, Calderon R 2007 Vertebral osteolysis after posterior interbody lumbar fusion with recombinant human bone morphogenetic protein 2: a report of five cases. Spine J 7:609-14.
10. Wong D A, Kumar A, Jatana S, Ghiselli G, Wong K 2008 Neurologic impairment from ectopic bone in the lumbar canal: a potential complication of off-label PLIF/TLIF use of bone morphogenetic protein-2 (BMP-2). Spine J 8:1011-8.
11. Smucker J D, Rhee J M, Singh K, Yoon S T, Heller J G 2006 Increased swelling complications associated with off-label usage of rhBMP-2 in the anterior cervical spine. Spine. 31:2813-9.
12. Carragee E J, Hurwitz E L, Weiner B K 2011 A critical review of recombinant human bone morphogenetic protein-2 trials in spinal surgery: emerging safety concerns and lessons learned. Spine J 11:471-91.
13. Kha H T, Basseri B, Shouhed D, Richardson J, Tetradis S, Hahn T J, Parhami F 2004 Oxysterols regulate differentiation of mesenchymal stem cells: pro-bone and anti-fat. J Bone Miner Res 19:830-40.
14. Kim W-K, Meliton V, Amantea C M, Hahn T J, Parhami F 2007 20(S)-hydroxycholesterol inhibits PPARgamma expression and adipogenic differentiation of bone marrow stromal cells through a Hedgehog-dependent mechanism. J Bone Miner Res 22:1711-9.
15. Johnson J S, Meliton V, Kim W K, Lee K-B, Wang J C, Nguyen K, Yoo D, Jung M E, Atti E, Tetradis S, Pereira R C, Magyar C, Nargizyan T, Hahn T J, Farouz F, Thies S, Parhami F 2011 Novel oxysterols have pro-osteogenic and anti-adipogenic effects in vitro and induce spinal fusion in vivo. J Cell Biochem 112:1673-84.
16. Parhami F, Morrow A D, Balucan J, Leitinger N, Watson A D, Tintut Y, Berliner J A, Demer L L 1997 Lipid oxidation products have opposite effects on calcifying vascular cell and bone cell differentiation. A possible explanation for the paradox of arterial calcification in osteoporotic patients. Arterioscler Thromb Vasc Biol 17:680-7.
17. Dwyer J R, Sever N, Carlson M, Nelson S F, Beachy P A, Parhami F 2007 Oxysterols are novel activators of the Hedgehog signaling pathway in pluripotent mesenchymal cells. J Biol Chem 282:8959-68.
18. Kim W-K, Meliton V, Bourquard N, Hahn T J, Parhami F 2010 Hedgehog signaling and osteogenic differentiation in multipotent bone marrow stromal cells are inhibited by oxidative stress. J Cell Biochem 111:1199-209.
19. Nachtergaele S, Mydock L K, Krishnan K, Rammohan J, Schlesinger P H, Covey D F, Rohatgi R 2012 Oxysterols are allosteric activators of the oncoprotein Smoothened. Nat Chem Biol 8:211-20.
20. Rohatgi R, Milenkovic L, Corcoran R B, Scott M P 2009 Hedgehog signal transduction by Smoothened: pharmacologic evidence for a 2-step activation process. Proc Natl Acad Sci USA 106:3196-201.
21. Alanay A, Chen C, Lee S, Murray S S, Brochmann E J, Miyazaki M, Napoli A, Wang J C 2008 The adjunctive effect of a binding peptide on bone morphogenetic protein enhanced bone healing in a rodent model of spinal fusion. Spine 33:1709-13.
22. Miyazaki M, Sugiyama O, Tow B, Zou J, Morishita Y, Wei F, Napoli A, Sintuu C, Lieberman J R, Wang J C 2008 The effects of lentiviral gene therapy with bone morphogenetic protein-2-producing bone marrow cells on spinal fusion in rats. J Spinal Disord Tech 21:372-9.
23. Pereira R C, Stadmeyer L E, Smith D L, Rydziel S, Canalis E 2007 CCAAT/Enhancer-binding protein homologous protein (CHOP) decreases bone formation and causes osteopenia. Bone 40:619-26.
24. Magyar C E, Aghaloo T L, Atti E, Tetradis S 2008 Ostene, a new alkylene oxide copolymer bone hemostatic material, does not inhibit bone healing. Neurosurgery 63:373-378; discussion 378.
25. Sintuu C, Simon R J, Miyazaki M, Morishita Y, Hymanson H J, Taghavi C, Brochmann E J, Murray S S, Wang J C 2011 Full-length spp24, but not its 18.5-kDa proteolytic fragment, inhibits bone-healing in a rodent model of spine fusion. J Bone Joint Surg Am 93:1022-32.
26. Miyazaki M, Morishita Y, He W, Flu M, Sintuu C, Hymanson H J, Falakassa J, Tsumura H, Wang J C 2009 A porcine collagen-derived matrix as a carrier for recombinant human bone morphogenetic protein-2 enhances spinal fusion in rats. Spine J 9:22-30.
27. Zhu W, Rawlins B A, Boachie-Adjei O, Myers E R, Arimizu J, Choi E, Lieberman J R, Crystal R G, Hidaka C 2004 Combined bone morphogenetic protein-2 and -7 gene transfer enhances osteoblastic differentiation and spine fusion in a rodent model. J Bone Miner Res 19:2021-32.

We claim:

1. A compound, Oxy149, having Formula I, 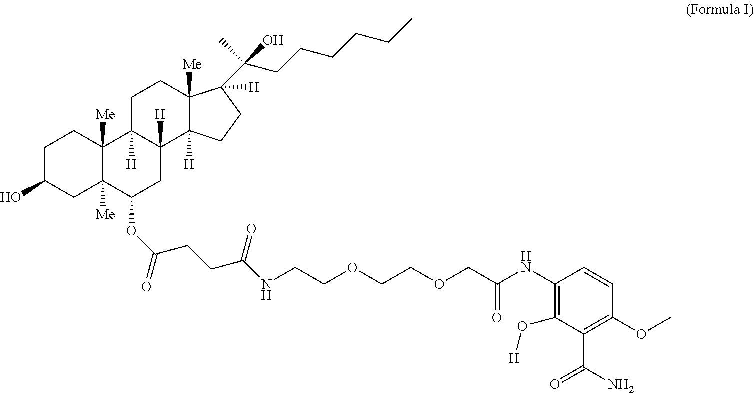
or a pharmaceutically acceptable salt or solvate thereof.

2. A bioactive composition, comprising the compound Oxy149 and a pharmaceutically acceptable carrier.

3. The bioactive composition of claim 2, further comprising at least one additional agent, selected from the group consisting of parathyroid hormone, sodium fluoride, insulin-like growth factor I (ILGF-I), insulin-like growth factor II (ILGF-II), transforming growth factor beta (TGF-β), a cytochrome P450 inhibitor, an osteogenic prostanoid, BMP 2, BMP 4, BMP 7, BMP 14 and an anti-resorptive agent.

4. A method for treating a subject having a bone disorder, osteoporosis, or a bone fracture, comprising administering to the subject an effective amount of the bioactive composition of claim 2.

5. The method of claim 4, comprising administering to the subject the bioactive composition at a therapeutically effective dose in an effective dosage form at a selected interval to increase bone mass.

6. The method of claim 4, comprising administering to the subject the bioactive composition at a therapeutically effective dose in an effective dosage form at a selected interval to ameliorate the symptoms of osteoporosis.

7. A method for treating a subject in need of an increase in osteomorphogenesis and/or osteoproliferation, comprising administering to the subject an effective amount of the composition of claim 2.

8. A method for treating a subject to induce bone formation, comprising administering the composition of claim 2 in an effective dosage form at a selected interval to increase bone mass.

9. A method for inducing osteoblastic differentiation of a mammalian mesenchymal stem cell, comprising contacting the cell with an effective amount of the composition of claim 2, wherein the mammalian mesenchymal stem cell is a marrow stromal cell in a subject.

10. A method for stimulating a hedgehog (Hh) pathway mediated response, in a cell or tissue in a subject, comprising contacting the cell or tissue with an effective amount of the bioactive composition of claim 2, wherein the Hh pathway mediated response is the stimulation of osteoblastic differentiation, osteomorphogenesis and/or osteoproliferation.

11. A method for treating a subject to induce bone formation comprising:
harvesting mammalian mesenchymal stem cells;
treating the mammalian mesenchymal cells with the bioactive composition of claim 2 to induce osteoblastic differentiation of the cells; and
administering the differentiated cells to the subject.

12. The method of any one of claims 4-10, wherein the bioactive composition is administered locally to a cell, tissue or organ in the subject.

13. The method of any one of claims 4-10, wherein the bioactive composition is administered to the subject systemically.

14. A method for stimulating a mammalian cell in a mammal to express a level of a biological marker of osteoblastic differentiation which is greater than the level of the biological marker in an untreated cell, comprising exposing the mammalian cell to an effective amount of the compound of claim 1.

15. The method of claim 14, wherein the biological marker is alkaline phosphatase activity, calcium incorporation, mineralization and/or expression of osteocalcin mRNA.

16. The method of claim 14, wherein the mammalian cell is a mesenchymal stem cell, an osteoprogenitor cell or a cell in a calvarial lesion, fracture or defect.

17. An implant for use in a human or animal body comprising a substrate having a surface, wherein the surface or the insides of the implant comprises the bioactive composition of claim 2 in an amount sufficient to induce bone formation in the surrounding bone tissue.

18. The implant of claim 17, wherein the substrate is formed into the shape of a pin, screw, plate, or prosthetic joint.
